(19) United States
(12) Patent Application Publication
(10) Pub. No.: US 2024/0360424 A1
(43) Pub. Date: Oct. 31, 2024

(54) ANION-EXCHANGE CHROMATOGRAPHY METHODS FOR PURIFICATION OF RECOMBINANT ADENO-ASSOCIATED VIRUSES

(71) Applicant: Ultragenyx Pharmaceutical Inc., Novato, CA (US)

(72) Inventors: Dennis Perry Chen, Boston, MA (US); Wenjun Di, Woburn, MA (US); Chao Huang, Belmont, MA (US); Ying Cai, Lexington, MA (US); James C. Warren, Hollis, NH (US)

(21) Appl. No.: 18/683,819

(22) PCT Filed: Aug. 17, 2022
(86) PCT No.: PCT/US2022/075056

§ 371 Date: Feb. 15, 2024

Related U.S. Application Data
(60) Provisional application No. 63/234,059, filed on Aug. 17, 2021.

(51) Int. Cl. C12N 7/00; B01D 15/36; B01D 15/42

(52) U.S. Cl. CPC C12N 7/00; B01D 15/363; B01D 15/426; C12N 2750/14123

(57) ABSTRACT

Provided are methods of separating full capsid particles and empty capsid particles in a viral capsid preparation using an anion exchange medium. Methods may involve using a wash solution comprising a quaternary ammonium salt. Methods include applying a viral capsid preparation to an anion exchange medium, combined with multiple quaternary ammonium salt wash solutions.

### CROSS-REFERENCE TO RELATED APPLICATIONS

The present application claims the benefit of and priority to U.S. Provisional Application No. 63/234,059 filed Aug. 17, 2021, the entire content of which is hereby incorporated by reference in its entirety for all purposes.

### BACKGROUND

A variety of gene therapy products are currently being developed to treat human diseases. Many of these therapeutics use recombinant adeno-associated viruses (rAAVs) that have been engineered to deliver a heterologous nucleic acid of interest (e.g., a gene encoding a therapeutic protein, an antisense nucleic acid molecule, a ribozyme, a miRNA, an siRNA, a nucleic acid encoding a CRISPR/Cas system, or the like) to the diseased target cells of a patient.

These rAAVs are engineered by deleting, in whole or in part, the internal portion of the AAV genome and inserting the heterologous nucleic acid of interest between the inverted terminal repeats (ITRs). The ITRs remain functional in such vectors allowing replication and packaging of the AAV particle containing the nucleic acid cargo enclosed within the AAV capsid. Typically, the heterologous nucleic acid is operably linked to regulatory sequences (e.g., promoter or enhancer) capable of driving expression of the cargo in the patient's target cells.

Various methods have been developed to manufacture large quantities of these rAAVs. These methods typically involve “upstream” operations where the rAAVs are produced within a host cell (e.g., a mammalian or insect cell) and “downstream” operations where the rAAVs are collected and purified. Typically, inefficiencies in the packaging of the nucleic acid cargo during the upstream phase lead to rAAV preparations that include a mixture of “full” AAV particles (i.e., particles that include the nucleic acid of interest) and “empty” AAV particles (i.e., AAV particles that lack in whole or in part the nucleic acid cargo).

The presence of empty AAV particles in a gene therapy product may increase the overall dose required to achieve therapeutic efficacy and may promote an immune response (e.g., development of neutralizing antibodies or T-cell activation) upon administration to a patient.

Therefore, there is a need in the art for downstream purification methods that can be used to separate full AAV particles from empty AAV particles in rAAV preparations that are produced by current upstream processes.

### SUMMARY

The present disclosure is based in part on the insight that the separation of full capsid particles and empty capsid particles in viral capsid preparations (e.g., rAAV preparations) using anion-exchange (AEX) chromatography can be improved when a quaternary ammonium (QA) chloride (e.g., tetraethylammonium chloride [TEAC]) is included in one or more of the solutions used for the separation; further improvements are seen particularly when used in conjunction with a magnesium salt (e.g., magnesium chloride [MgCl₂]).

In one aspect, provided are methods of separating full capsid particles and empty capsid particles in a viral capsid preparation, the methods comprising:
a) applying the viral capsid preparation to an anion exchange medium;
b) passing a first wash solution comprising a quaternary ammonium salt through the anion exchange medium to obtain a wash fraction comprising empty viral capsid particles;
c) passing a second wash solution through the anion exchange medium to obtain a second wash fraction comprising the quaternary ammonium salt;
d) passing an elution solution through the anion exchange medium to elute the full capsid particles; and
e) collecting an elution fraction comprising full capsid particles;
   thereby separating full capsid particles and empty capsid particles,
      wherein (i) the concentration of the quaternary ammonium salt in the first wash solution remains constant throughout step (b) and/or (ii) the elution solution's salt composition remains constant throughout step (d).

In some embodiments, the methods comprise repeating one or more of step b), step c), and step d) one or more times.

In one aspect, provided are elution methods comprising:
(a) contacting an anion-exchange medium with a viral capsid preparation comprising full capsid particles and empty capsid particles;
(b) applying to the anion-exchange medium a first wash solution comprising a quaternary ammonium salt to obtain a first wash fraction;
(c) applying to the anion-exchange medium a second wash solution, to obtain a second wash fraction; and
(d) applying to the anion-exchange medium an elution solution, and collecting an elution fraction,
wherein (i) the concentration of the quaternary ammonium salt in the first wash solution remains constant throughout step (b) and/or (ii) the elution solution's salt composition remains constant throughout step (d).

In some embodiments, the methods comprise repeating one or more of step b), step c), and step d) one or more times.

In some embodiments, the methods comprise, the second wash solution and the elution solution do not comprise a quaternary ammonium salt.

In some embodiments, aside from the first wash solution, no other solution comprising the quaternary ammonium salt is passed through or applied to the anion exchange medium.

In some embodiments, aside from the first wash solution, no other solution comprising any quaternary ammonium salt is passed through or applied to the anion exchange medium.

In some embodiments, the first wash solution comprises a quaternary ammonium salt at a concentration from about 90 mM to about 130 mM, e.g., about 110 mM.

In some embodiments, the quaternary ammonium salt is a tetraalkylammonium chloride. For example, in some embodiments, the tetraalkylammonium chloride is selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof. In some embodiments, wherein the tetraalkylammonium chloride is TEAC. In some embodiments, the quaternary ammonium salt is a tetraalkylammonium acetate.

In some embodiments, the quaternary ammonium salt is selected from the group consisting of tetramethylammonium acetate, tetraethylammonium acetate (TEA-Ac), tetrapropylammonium acetate, tetrabutylammonium acetate, and any combination(s) thereof. For example, in some embodiments, wherein the quaternary ammonium salt is TEA-Ac.

In some embodiments, the quaternary ammonium salt is choline chloride.

In some embodiments, any one or more of the first wash solution, the second wash solution, and/or the elution solution further comprise a divalent salt.

In some embodiments, the first wash solution further comprises a divalent salt, e.g., MgCl₂. In some embodiments, the first wash solution comprises MgCl₂ at a concentration from about 1 mM to about 10 mM, e.g., about 2 mM.

In some embodiments, the first wash solution comprises TEAC at a concentration from about 30 mM to about 200 mM, e.g., about 110 mM. In some such embodiments, the first wash solution further comprises a divalent salt, e.g., MgCl₂. In some embodiments, the first wash solution comprises MgCl₂ at a concentration from about 1 mM to about 10 mM, e.g., about 2 mM.

In some embodiments, the second wash solution and/or the elution solution comprises NaCl, Na₂SO₄, MgSO₄, or any combination thereof. In some embodiments, the second wash solution comprises NaCl, e.g., at a concentration from about 25 mM to about 375 mM, from about 50 mM to about 250 mM, from about 70 mM to about 200 mM, from about 70 mM to about 140 mM, or from about 90 mM to about 140 mM. In some embodiments, the elution solution comprises NaCl, e.g., at a concentration from about 25 mM to about 375 mM, from about 50 mM to about 250 mM, from about 70 mM to about 200 mM, from about 70 mM to about 140 mM, or from about 90 mM to about 140 mM.

In some embodiments, the elution solution comprises an anionic species, e.g., an anionic species is selected from the group consisting of tetrafluoroborate (BF₄), bromide (Br), and acetate (Ac).

In some embodiments, the elution solution comprises MgCl₂, e.g., MgCl₂ at a concentration of at least 1 mM, about 1 mM to about 5 mM, about 1 mM, about 2 mM, about 3 mM, about 4 mM, or about 5 mM.

In some embodiments, the first wash solution, the second wash solution, and/or the elution solution each comprise a buffer system at a pH of about 9.

In some embodiments, the first wash solution elutes at least 60% or at least 90% (e.g., from 90% to 100%, such as about 90%, 91%, 92%, 93%, 94%, 95%, 96%, 97%, 98%, 99%, or 100%) of the empty capsid particles present in the viral capsid preparation. In some embodiments, the first wash solution does not substantially elute the full capsid particles present in the viral capsid preparation. In some embodiments, the first wash solution elutes 30% or less (e.g., from 0 to 30%, such as about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%) or 1% or less of the full capsid particles present in the viral capsid preparation.

In some embodiments, the second wash fraction comprises the quaternary ammonium salt. In some embodiments, the second wash solution elutes at least 50%, at least 90% (e.g., from 90% to 100%, such as about 90%, 91%, 92%, 93%, 94%, 95%, 96%, 97%, 98%, 99%, or 100%), or at least 99% of the quaternary ammonium salt present in the first wash solution.

In some embodiments, the second wash solution does not substantially elute the full capsid particles present in the viral capsid preparation. In some embodiments, the second wash solution elutes 20% or less (e.g., from 0 to 20%, such as about 0, 1%, 2%, 3%, 4%, 5%, 6%, 7%, 8%, 9%, 10%, 11%, 12%, 13%, 14%, 15%, 16%, 17%, 18%, 19%, or 20%) or 1% or less of the full capsid particles present in the viral capsid preparation.

In some embodiments, the elution solution substantially elutes the full capsid particles present in the viral capsid preparation. In some embodiments, the elution solution elutes at least 50%, at least 90%, at least 99% of the full capsid particles present in the viral capsid preparation.

In some embodiments, the elution fraction comprises at least 50% (e.g., from 50% to 100%, such as about 50%, 55%, 60%, 65%, 70%, 75%, 80%, 85%, 90%, 95%, or 100%), at least 90% (e.g., from 90% to 100%, such as about 90%, 91%, 92%, 93%, 94%, 95%, 96%, 97%, 98%, 99%, or 100%) of, or at least 99% of the full capsid particles present in the viral capsid preparation.

In some embodiments, the elution fraction comprises 50% or less, 10% or less, or 1% or less of the empty capsid particles present in the viral capsid preparation, thereby substantially purifying the full capsid particles from the viral capsid preparation.

In some embodiments, at least 35% (e.g., from 35% to 100%, such as about 35%, 40%, 45%, 50%, 55%, 60%, 65%, 70%, 75%, 80%, 85%, 90%, 95%, or 100%) of the capsid particles present in the elution fraction are full capsid particles.

In some embodiments, at least 40% (e.g., from 40% to 100%, such as about 40%, 45%, 50%, 55%, 60%, 6%, 70%, 7%, 80%, 85%, 90%, 95%, or 100%) of the capsid particles present in the elution fraction are full capsid particles.

In some embodiments, at least 45% (e.g., from 45% to 100%, such as about 40%, 45%, 50%, 55%, 60%, 65%, 70%, 75%, 80%, 85%, 90%, 95%, or 100%) of the capsid particles present in the elution fraction are full capsid particles.

In some embodiments, at least 50% (e.g., from 50% to 100%, such as about 50%, 55%, 60%, 65%, 70%, 75%, 80%, 85%, 90%, 95%, or 100%) of the capsid particles present in the elution fraction are full capsid particles.

In some embodiments, more than 50% (e.g., more than 50% and up to 100%, such as about 51%, 55%, 60%, 65%, 70%, 75%, 80%, 85%, 90%, 95%, or 100%) of the capsid particles present in the elution fraction are full capsid particles.

In certain embodiments, the concentration of the quaternary ammonium salt in the first wash solution remains constant throughout step (b). In some embodiments, the first wash solution's salt composition remains constant throughout step (b). In some embodiments, the second wash solution's salt composition remains constant throughout step (c).

In some embodiments, the elution solution's salt composition remains constant throughout step (d). In some embodiments, from 1 to 20 total column volumes, e.g., from 1 to 10 total column volumes are eluted.

In some embodiments, the first wash solution's salt composition, the second wash solution's salt composition, and the elution solution's salt composition are remaining constant throughout each individual wash or elution step.

In some embodiments, the first wash solution's composition varies during step (b).

In some embodiments, the second wash solution's composition varies during step (c). For example, in some embodiments, the concentration of a salt within the second wash solution increases continuously over time throughout step (c). In some embodiments, this continuous increase is linear over time throughout step (c).

In some embodiments, the elution solution's composition varies during step (d). For example, in some embodiments, the concentration of a salt within the elution solution increases continuously over time throughout step (d). In some embodiments, this continuous increase is linear over time throughout step (d)

In some embodiments, 0-200 total column volumes, e.g., from 50 to 150 total column volumes, e.g., about 90 total column volumes are eluted.

In some embodiments, the capsid is from AAV8 or a variant thereof.

In some embodiments, the anion exchange medium is a monolithic column. For example, in some embodiments, the monolithic column is a CIMmultus® QA column.

In some embodiments, the method does not comprise using an isocratic elution gradient of MgCl₂.

In some embodiments, if one or more of the first wash solution comprises MgCl₂, and the concentration of MgCl₂ in the first wash solution is constant, the concentration of MgCl₂ is only constant throughout step (b).

In one aspect, provided are methods of separating full capsid particles and empty capsid particles in a viral capsid preparation, the methods comprising:
(a) applying the viral capsid preparation to an anion exchange medium;
(b) passing a first wash solution comprising a quaternary ammonium salt through the anion exchange medium to obtain a wash fraction comprising empty viral capsid particles;
(c) passing a second wash solution through the anion exchange medium to obtain a second wash fraction comprising the quaternary ammonium salt;
(d) passing an elution solution through the anion exchange medium to elute the full capsid particles; and
(e) collecting an elution fraction comprising full capsid particles;
thereby separating full capsid particles and empty capsid particles,
wherein (i) the concentration of the quaternary ammonium salt in the first wash solution remains constant throughout step (b),
wherein the capsid is from AAV8 or a variant thereof, and
wherein at least 35% (e.g., from 35% to 100%, such as about 35%, 40%, 45%, 50%, 55%, 60%, 65%, 70%, 75%, 80%, 85%, 90%, 95%, or 100%) of the capsid particles present in the elution fraction are full capsid particles.

In some embodiments, the quaternary ammonium salt is a tetraalkylammonium chloride, e.g., a tetraalkylammonium chloride is selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof. In some embodiments, the tetraalkylammonium chloride is TEAC.

In some embodiments, the quaternary ammonium salt is a tetraalkylammonium acetate, e.g., a tetraalkylammonium acetate selected from the group consisting of tetramethylammonium acetate, tetraethylammonium acetate (TEA-Ac), tetrapropylammonium acetate, tetrabutylammonium acetate, and any combination(s) thereof. In some embodiments, the quaternary ammonium salt is TEA-Ac.

In some embodiments, the quaternary ammonium salt is choline chloride.

In one aspect, provided are methods for manufacturing full recombinant adeno-associated virus (rAAV) capsid particles, the methods comprising the steps of.
(a) producing a viral capsid preparation comprising full rAAV capsid particles and empty rAAV capsid particles from cultured mammalian or insect cells;
(b) applying the viral capsid preparation to an anion exchange medium;
(c) passing a first wash solution comprising a quaternary ammonium salt through the anion exchange medium to obtain a wash fraction comprising empty rAAV viral capsid particles;
(d) passing a second wash solution through the anion exchange medium to obtain a second wash fraction comprising the quaternary ammonium salt;
(e) passing an elution solution through the anion exchange medium to elute the full rAAV capsid particles; and
(f) collecting an elution fraction comprising full rAAV capsid particles;
wherein (i) the concentration of the quaternary ammonium salt in the first wash solution remains constant throughout step (c) and/or (ii) the elution solution's salt composition remains constant throughout step (e).

In one aspect, provided are methods for manufacturing full recombinant adeno-associated virus (rAAV) capsid particles, the methods comprising the steps of:
(a) producing a viral capsid preparation comprising full rAAV capsid particles and empty rAAV capsid particles from cultured mammalian or insect cells;
(b) contacting an anion-exchange medium with the viral capsid preparation;
(c) applying to the anion-exchange medium a first wash solution comprising a quaternary ammonium salt to obtain a first wash fraction;
(d) applying to the anion-exchange medium a second wash solution, to obtain a second wash fraction; and
(e) applying to the anion-exchange medium an elution solution, and collecting an elution fraction,
wherein (i) the concentration of the quaternary ammonium salt in the first wash solution remains constant throughout step (c) and/or (ii) the elution solution's salt composition remains constant throughout step (e),
wherein the elution fraction comprises full rAAV capsid particles.

In some embodiments, the concentration of the quaternary ammonium salt in the first wash solution remains constant throughout step (c)

In some embodiments, the rAAV capsid particles are rAAV8 capsid particles.

In some embodiments, the quaternary ammonium salt is a tetraalkylammonium chloride, e.g., a tetraalkylammonium chloride selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof. In some embodiments, the quaternary ammonium salt is TEAC.

In some embodiments, the quaternary ammonium salt is a tetraalkylammonium acetate, e.g., a tetraalkylammonium acetate selected from the group consisting of tetramethylammonium acetate, tetraethylammonium acetate (TEA-Ac), tetrapropylammonium acetate, tetrabutylammonium acetate, and any combination(s) thereof. In some embodiments, the quaternary ammonium salt is TEA-Ac.

In some embodiments, the quaternary ammonium salt is choline chloride.

In some embodiments, at least 35% (e.g., 35% to 100%, such as about 35%, 40%, 45%, 50%, 55%, 60%, 65%, 70%, 75%, 80%, 85%, 90%, 95%, or 100%) of rAAV capsid particles in the elution fraction are full rAAV capsid particles.

In one aspect, provided are compositions comprising full recombinant adeno-associated virus (rAAV) capsid particles, produced by a method of manufacture as disclosed herein. In some embodiments, at least 35% (e.g., 35% to 100%, such as about 35%, 40%, 45%, 50%, 55%, 60%, 65%, 70%, 75%, 80%, 85%, 90%, 95%, or 100%) of rAAV capsid particles in the composition are full rAAV capsid particles.

### BRIEF DESCRIPTION OF THE DRAWING

FIGS. 1A and 1B depict exemplary chromatograms demonstrating separation of empty AAV particles from full AAV8 particles using a NaCl gradient on various types of stationary phases. FIG. 1A depicts chromatograms from experiments on AAV8-1 particles, using either a POROS™ 50 HQ column or a CIM-Q column. FIG. 1B depicts chromatograms from experiments on AAV8-2 particles, using either a Nuvia™ Q column or a CIM-Q column. “E” denotes fractions corresponding to high relative amounts of empty capsids, and “F” denotes fractions corresponding to high relative amounts of full capsids. The rising straight lines show the increase in solution conductivity caused by the corresponding increase in NaCl concentration. (See Example 1.)

FIGS. 2A and 2B depict results from separation experiments run on AAV8-2 material on NaCl gradients, run on other Nuvia™ Q columns or CIMmultus® QA columns. (See Example 1.) FIG. 2A depicts the step yield, the amount of genome of interest in the pooled fraction relative to the amount in load material, as determined by quantitative polymerase chain reaction (qPCR) methods. FIG. 2B depicts the percentage of full capsid particles present in eluates, as determined by analytical ultra-centrifugation (AUC). Values in both FIGS. 2A and 2B are normalized to those observed with the Nuvia™ Q columns.

FIGS. 3A-3E depict exemplary chromatograms from separation experiments run on human embryonic kidney (HEK)-produced AAV8-1 (FIG. 3A), AAV8-2 (FIG. 3B), AAV8-3 (FIG. 3C), and hu37 (FIG. 3D) and on HeLa-produced AAV9 (FIG. 3E). Chromatography was performed in CIMmultus® QA columns using a NaCl gradient. The rising straight lines show the increase in solution conductivity caused by the corresponding increase in NaCl concentration. (See Example 2.)

FIG. 4 depicts graphs comparing results from experiments run on HEK-produced AAV8-1 and AAV8-2. Shown are the normalized percentages of full capsid particles in eluate (y-axis) as compared to percentages of full capsid particles in the load material (x-axis). (See Example 2.)

FIG. 5 depicts a flow chart outlining downstream processing of AAV material produced from HeLa cells (left-hand side of FIG. 5), and representative chromatograms (right-hand side of FIG. 5) from experiments in which samples containing no MgCl₂, 67 mM MgCl₂ (“no dilution in load”), or 6-7 mM MgCl₂ (“dilution in load”) were subject to NaCl gradient elution conditions on POROS™ XQ columns. “E” denotes fractions corresponding to high relative amounts of empty capsids, and “F” denotes fractions corresponding to high relative amounts of full capsids. (See Example 3.)

FIG. 6 depicts exemplary chromatograms demonstrating increased empty AAV particle to full AAV particle separation for a quaternary ammonium (QA) salt gradient, in this example, tetramethylammonium chloride (TMAC) (bottom chromatogram), in comparison to a NaCl gradient (top chromatogram). The chromatograms depict A₂₅₄ and A₂₈₀ measurements of eluate during a gradient elution. The rising straight line shows the increase in solution conductivity caused by the corresponding increase in eluent concentration. (See Example 4.)

FIGS. 7A-7D depict exemplary chromatograms demonstrating an increase in empty AAV particle to full AAV particle separation for QA salt gradients with increasing sizes of functional groups. From top to bottom, the elution is obtained from a sodium chloride (NaCl) (FIG. 7A), tetramethylammonium chloride (TMAC) (FIG. 7B), tetrabutylammonium chloride (TBAC) (FIG. 7C), and benzyltributylammonium chloride (BTBAC) (FIG. 7D) gradient. A chromatogram of a NaCl gradient is depicted as a reference for comparing “empty” and “full”-fraction resolution. The chromatograms depict A₂₈₀ measurements of eluate during a gradient elution. The rising straight line shows the increase in solution conductivity caused by the corresponding increase in eluent concentration. (See Example 5.)

FIG. 8 depicts a graph plotting ionic strength (x-axis) of the first and second peaks as a function of the size of positive ions from the separation experiments described in Example 6, which use QA salt gradients with increasing sizes of functional groups. (See Example 5.)

FIG. 9 depicts an exemplary chromatogram depicting separation of empty- and full-AAV particles using a gradient elution with an about 25 mM to about 337.5 mM tetraethylammonium chloride (TEAC) gradient on a CIMmultus® QA column over 90 column volumes at a constant magnesium chloride (MgCl₂) concentration of 2 mM at pH 9. The rising straight line shows the increase in solution conductivity caused by the corresponding increase in TEAC concentration. (See Example 6.)

FIG. 10 depicts an exemplary chromatogram depicting separation of empty and full AAV particles using a gradient elution involving an initial wash with tetraethylammonium acetate (TEA-Ac) and constant MgCl₂ concentration. The rising straight line shows the increase in solution conductivity caused by the corresponding increase in TEA-Ac concentration. (See Example 6.)

FIG. 11 depicts an exemplary chromatogram depicting separation of empty and full AAV particles using an isocratic elution involving an initial wash with tetraethylammonium chloride (TEAC) and magnesium chloride (MgCl₂), followed by two sequential step washes with sodium chloride (NaCl) and MgCl₂, and followed by elution with NaCl and MgCl₂. The dashed line corresponds to the volumetric output of pump “B” (i.e., the pump(s) that feed in wash and elution buffers) relative to pump “A” (i.e., the pump(s) that feed in equilibration buffers). (See Example 8.)

FIGS. 12A-12E depict exemplary chromatograms from experiments using a variety of protocols, using MgSO₄, to separate empty and full AAV8-2 particles. Shown from top to bottom are chromatograms from experiments using a CIMmultus® QA column and 1) gradient elution (FIG. 12A); 2) gradient wash, followed by isocratic elution (FIG. 12B); 3) single isocratic wash (FIG. 12C); 4) isocratic elution with three washes (FIG. 12D); or 5) isocratic elution with two washes (FIG. 12E). Orange lines on each chromatogram show the solution conductivity corresponding to the salt concentration. (See Example 9.)

FIGS. 13A and 13B depict results from separation experiments run on AAV8-1 material using gradient elution or isocratic elution protocols on CIMmultus® QA columns. (See Example 10.) FIG. 13A depicts the step yield, the amount of genome of interest in the pooled fraction relative to the amount in load material, as determined by quantitative polymerase chain reaction (qPCR) methods. FIG. 13B depicts the percentage of full capsid particles present in eluates, as determined by analytical ultra-centrifugation (AUC). Values in both FIGS. 13A and 13B are normalized to those observed with the gradient elution protocols. (See Example 9.)

FIG. 14 depicts an exemplary chromatogram depicting separation of empty and full AAV particles using a gradient elution involving an initial wash with choline chloride and constant MgCl₂ concentration. The rising straight line shows the increase in solution conductivity caused by the corresponding increase in choline chloride concentration. (See Example 10.)

FIG. 15A depicts the parameters for the five different runs tested in a set of experiments described in Example 11. Columns were loaded with 1×, 6×, and 12× amount of AAV8-2 material, with a residence time of 1×, (½)×, or (¼)×. Samples were run on a CIMmultus® QA column using an NaCl gradient. FIG. 15B depicts chromatograms from the various runs whose conditions are shown in FIG. 15A. “E” denotes fractions corresponding to high relative amounts of empty capsids, and “F” denotes fractions corresponding to high relative amounts of full capsids. (See Example 11.)

FIG. 15C shows plots from a desirability analysis using JMP® software, for variables such as capsid loading, normalized host cell protein amounts in eluate, and normalized percentages of full capsids in eluates. (See Example 11.)

FIGS. 16A and 16B show the yields (of genome of interest), percentages of full capsids, and host cell protein amounts from chromatography experiments using AAV8-1 material run on CIMmultus® QA columns using an NaCl gradient. FIG. 16A shows amounts by fraction, and FIG. 16B shows cumulative amounts. As can be seen in FIGS. 16A and 16B, fractions after the second peak have detectable host cell protein levels, and the collecting additional fractions to increase the percentage of full capsid proteins collected also increases the cumulative amounts of host cell protein.

FIG. 17 depicts representative chromatograms from experiments on samples of AAV8-1 and AAV8-2 material comprising full and empty capsid particles, run on CIMmultus® QA columns using either an NaCl gradient or a gradient with a mobile phase comprising MgSO₄. (See Example 12.)

FIGS. 18A-18C depict representative chromatograms from experiments on samples of AAV8.1 material comprising full and empty capsid particles, run on CIMmultus® QA columns using one of three conditions: 1) NaCl, with gradient elution (FIG. 18A); 2) tetraethylammoniumchloride (TEAC) gradient and 2 mM MgCl₂ (FIG. 18B); and 3) tetraethylammoniumchloride (TEAC) and 2 mM MgCl₂, with isocratic elution (FIG. 18C). “E” denotes fractions corresponding to high relative amounts of empty capsids, and “F” denotes fractions corresponding to high relative amounts of full capsids. (See Example 13.)

FIGS. 19A and 19B depict plots that show step yields (genomes of interest as measured by quantitative PCR) and percentages of full capsid particles (as measured by analytical ultracentrifugation) from these experiments, normalized to the levels for the NaCl gradient experiments. (See Example 13.)

FIG. 20 depicts representative chromatograms from experiments on samples of AAV8.1 material comprising full and empty capsid particles, run on CIMmultus® QA columns using one of several linear-gradient elution solutions comprising anionic species: tetraethylammonium (TEA)-BF4 (top panel), TEA-Br (middle panel), or TEA-Ac (bottom panel). Within each chromatogram, solid lines represent absorbance at 280 nm (A₂₈₀), and dashed lines represent ionic strength. (See Example 14.)

FIG. 21 depicts representative chromatograms from experiments on samples of AAV8.1 material comprising full and empty capsid particles, run on CIMmultus® QA columns using various concentrations (0, 0.2 mM, 1 mM, and 5 mM) of MgCl₂ in the elution solution. Traces (solid lines) correspond to absorbance at 280 nm (A₂₈₀), and asterisks denote a third population not apparently visible in traces from runs using elution solution containing 1 mM or 5 mM MgCl₂. Particle titers measured in the elution pool of the “Empty” peak (left-most peak in each chromatogram) are displayed adjacent to their corresponding elution profiles. (See Example 15.)

FIGS. 22A-22D depict representative chromatograms from experiments on samples of AAV8.1 material comprising full and empty capsid particles, run on CIMmultus® QA columns using a gradient elution protocol (FIGS. 22A and 22B) or an isocratic elution protocol (FIGS. 22C and 22D). FIGS. 22B and 22D depict magnified images of the peaks from FIGS. 22A and 22C, respectively. Within each chromatogram, light solid lines correspond to absorbance at 280 nm (A₂₈₀), and heavy solid lines correspond to absorbance at 254 nm (A₂₅₄)/Dashed lines in FIGS. 22A and 22C correspond to the volumetric fraction of running buffer B (20 mM bis-tris propane, 0.001% (w/v) Pluronic® F-68, 2 mM MgCl₂, and 200 mM NaCl at pH 9) pumped through the system. Dashed lines in FIGS. 22B and 22D correspond to the solution conductivity. (See Example 16.)

FIG. 23 depicts regression plots of the trade-off between step yield (genome recovery %) (y-axis) against percentages of full capsid particles (x-axis) obtained from anion exchange chromatography runs using NaCl in process buffers (open markers) or anion exchange chromatography runs using an isocratic TEA-Ac wash. Circles denote data from runs that used isocratic elution, while squares denote data from runs that used gradient elution. (See Example 17.)

### DETAILED DESCRIPTION OF CERTAIN EMBODIMENTS

The present disclosure provides improved methods for separating populations within a viral capsid preparation, e.g., for separating full capsid particles from empty capsid particles. Also provided are methods for manufacturing full recombinant adeno-associated virus (rAAV) capsid particles.

### Definitions

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

As used herein, the term “adeno-associated virus” refers to a small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 13 recognized serotypes of AAV (AAV1-13).

Unless otherwise noted, where the term “between” is used to refer to a numerical range, the range includes the specified endpoints. For example, the range “between 1 mM and 10 mM” includes 1 mM, 10 mM, and values greater than 1 mM but less than 10 mM.

As used herein, the term “capsid particle” refers to a particle that comprises at least one viral capsid protein which (i) encapsidates a nucleic acid, e.g., a vector genome or a portion thereof, and/or (ii) forms a structure surrounding a core. In the case of empty capsid particles, as described herein, the core may be empty or collapsed, or may contain only a portion of a vector genome. In some embodiments, a capsid particle encapsidates a nucleic acid that is a vector genome and/or gene of interest. In some embodiments, a capsid particle encapsidates a nucleic acid species that is not a vector genome or gene of interest, e.g., plasmid or host cell DNA, or a portion thereof.

As used herein, the term “full capsid particle” refers to a capsid particle that comprises a complete vector genome, that is, a vector genome that comprises a heterologous nucleic acid of interest flanked on both sides by AAV ITRs.

As used herein, the term “empty capsid particle,” refers to a capsid particle that includes at least one capsid protein and lacks a complete vector genome, e.g., the lacks in whole or in part, a heterologous nucleic acid of interest flanked on either side by AAV ITRs, or lacks in whole or in part, another part of the vector genome.

Unless otherwise noted, where the terms “from” and “to” are used to refer to a numerical range, the range includes the specified endpoints. For example, the range “from 1 mM to 10 mM” includes 1 mM, 10 mM, and values greater than 1 mM but less than 10 mM, and all integers including and in between 1 mM to 10 mM.

As used herein, the terms “gradient elution” or “gradient separation” refer to a mode of chromatographic separation wherein the concentration of one or more salts in the elution solution that is applied to the separation medium is gradually changed during the separation.

As used herein, the term “inverted terminal repeat” (abbreviated “ITR”) refers to a symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV integrating vectors.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the terms “isocratic elution” or “isocratic separation” refer to a mode of chromatographic separation where the concentration of all salts in the solution is kept constant during a defined period of the separation (e.g., the “wash” solution during a “wash” step and the “elution” solution during an “elution” step). In some embodiments, an isocratic elution uses a series of two or more separate solutions during the separation, each of which may have different fixed concentrations of one or more salts relative to another solution in the series.

As used herein, the phrase “isocratic elution gradient” refers to a gradient wherein the composition of the mobile phase is changed in steps during a single chromatographic run. In each step of an isocratic elution gradient, the mobile phase is kept at the same composition (e.g., constant concentration) until a subsequent step in the chromatographic run, at which time the composition of the mobile phase is changed such that the concentration of a component of the mobile phase is increased relative to the concentration of the component in a previous step. Thus, for example, an isocratic elution gradient of MgCl₂ involves using various steps, with a constant MgCl₂ concentration at each individual step, but with increasing MgCl₂ concentrations from one step to a subsequent step.

As used herein, “obtaining” or “to obtain” a fraction, e.g., a wash fraction after a step of passing through or applying a solution to an anion exchange medium, it is meant that the fraction is generated after that step. A fraction that is “obtained” may or may not be collected.

As used herein, the term “quaternary ammonium salt” refers to an ionic compound having a quaternary ammonium nitrogen, four groups (e.g., alkyl or aryl groups) connected to the ammonium nitrogen, and an anionic ion (e.g., acetate, bromide, or chloride).

As used herein, the term “recombinant,” may be used to describe, e.g., a nucleic acid molecule that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques.

A “recombinant adeno-associated virus preparation” or “rAAV preparation,” refers to a product that results from a method of producing (e.g., manufacturing) recombinant AAV in a host cell (e.g., in a mammalian cell or an insect cell). In some embodiments, a recombinant AAV preparation includes a mixture of full AAV particles and empty AAV particles. In some embodiments, a recombinant AAV preparation has been subjected to one or more downstream operations after initial upstream operations, e.g., nuclease treatment, filtration to remove host-cell impurities, and/or affinity purification using ligands that bind AAV capsids, as well known to those of skill in the art.

As used herein, the term “salt composition,” when used in reference to a solution, refers to the identities and amounts of all salts in that solution. Thus, if a solution's “salt composition” is said to remain constant throughout a particular duration, it is meant that the identities and amounts of all salts in that solution remain constant throughout the particular duration.

As used herein, the term “separation chemistry” refers to the active ligand, such as quaternary amine or mixed and supporting matrix of the separation medium.

As used herein, the term “separation medium” refers to a physical structure, such as column packed with resins or a monolith or a membrane, to which a rAAV preparation is applied in order to achieve separation of certain fractions of the preparation. For example, a rAAV preparation may be applied to a column, which column is then washed with one or more solutions to separate (and collect separated fractions) empty and full-AAV particles from one another. In some embodiments, a separation medium is an anion-exchange medium. In some embodiments, a separation medium is a mixed-modal medium that can serve as an anion-exchange medium. In some embodiments, a separation medium is a column (e.g., a monolithic column or particles in a packed column). In some embodiments, a separation medium is a membrane.

As used herein, the term “vector” is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes. In some embodiments herein, the vector is an AAV vector.

### Viral Capsid Preparations

Methods of the present disclosure are useful for separating full capsid particles and empty capsid particles in a viral capsid preparation, e.g., a viral capsid preparation that results from a process intended to generate a recombinant virus comprising a heterologous nucleic acid.

### Production of Viral Capsid Preparations

Viral capsid preparations can be produced (e.g., “manufactured”) from cultured host cells. A vast range of host cells can be used, such as bacteria, yeast, insect, or mammalian cells, etc. In some embodiments, the cultured host cells are mammalian or insect host cells. In some embodiments, the host cell can be a cell (or a cell line) appropriate for production or manufacture of recombinant AAV (rAAV), for example, a HeLa, Cos-7, HEK293, A549, BHK, Vero, RD, HT-1080, ARPE-19, or MRC-5 cell. In some embodiments, the host cell is a HeLa cell. In some embodiments, the host cell is a HEK293 cell.

Recombinant nucleic acid molecules or vectors (e.g., recombinant AAV vectors) can be delivered into the host cell culture using any suitable method known in the art. In some embodiments, a stable host cell line that has the recombinant nucleic acid molecule or vector inserted into its genome is generated. In some embodiments, a stable host cell line is generated, which contains an AAV vector described herein. After transfection of the AAV vector to the host culture, integration of the rAAV into the host genome can be assayed by various methods, such as antibiotic selection, fluorescence-activated cell sorting, southern blot, PCR based detection, fluorescence in situ hybridization as described by Nakai et al., Nature Genetics (2003) 34, 297-302; Philpott et al., Journal of Virology (2002) 76(11):5411-5421, and Howden et al., J Gene Med 2008; 10:42-50. Furthermore, a stable cell line can be established according to protocols well known in the art, such as those described in Clark, Kidney International Vol 61 (2002):S9-S15, and Yuan et al., Human Gene Therapy 2011 May; 22(5):613-24.

In producing viral capsid preparations, host cells are typically supplied with viral vectors. For example, for producing (e.g., manufacturing) AAV capsid preparations, host cells may be supplied with AAV vectors, Rep and Cap gene functions, and additional helper functions. Rep and Cap gene functions can be provided to the host cell by various means, for example, by a plasmid or any type of vector containing the wild-type AAV Rep and Cap genes, and electroporation of Rep and Cap mRNAs. Additional helper functions can be provided by, for example, an adenovirus (AV) infection, by a plasmid that carries all of the required AV helper function genes, or by other viruses such as herpes simplex virus (HSV) or baculovirus. Any genes, gene functions, or other genetic material necessary for rAAV production by the host cell may transiently exist within the host cell, or be stably inserted into the host cell genome. rAAV production methods suitable to produce viral capsid preparations include those disclosed in Clark et al., Human Gene Therapy 6:1329-1341 (1995), Martin et al., Human Gene Therapy Methods 24:253-269 (2013), Thorne et al., Human Gene Therapy 20:707-714 (2009), Fraser Wright, Human Gene Therapy 20:698-706 (2009), and Virag et al., Human Gene Therapy 20:807-817 (2009).

To release viral capsids from host cells, a variety of methods may be used. For example, lysis of virus-infected cells (e.g., AAV-infected cells) can be accomplished by methods that chemically or enzymatically treat the cells in order to release infectious viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used.

Alternatively, supernatant may be collected from AAV-infected cells without the need for cell lysis.

After release of viral capsids, samples may be subject to one or more processes before being used with methods of the currently disclosure, such as purification to remove cellular debris and/or helper virus particles, and/or heat inactivation of helper virus.

### Characteristics of Viral Capsid Preparations

Typically, the full and empty capsid particles in a given viral capsid preparation are capsid particles of the same virus and same serotype. In some embodiments, the capsid particles comprise a capsid of an adeno-associated virus (AAV) such as a recombinant AAV (rAAV). Examples of AAV capsid serotypes include serotypes 8, 1, 2, 3, 4, 5, 6, 7, 9, 10, 11, 12, 13, rh10, hu37, or a variant of any one thereof. In some embodiments, the capsid is from AAV8 or a variant thereof. In some embodiments, the capsid is from AAV9 or a variant thereof. In some embodiments, the capsid is from AAVrh10 or a variant thereof. In some embodiments, the capsid is from AAVhu37 or a variant thereof.

### Anion-Exchange Media

The methods of the present disclosure are not limited to any particular column architecture or dimensions, type of separation medium, or type of separation chemistry. For example, in some embodiments, the anionic exchange medium is a weak ionic exchanger. In some embodiments, the anionic exchange medium is a strong ionic exchanger.

In some embodiments, the anion exchange medium is in the form of a packed bed.

In some embodiments, the anion exchange medium is a chromatographic monolithic column. As but one example, a CIMmultus® monolithic column (e.g., CIMmultus® monolithic QA column) may be used.

### Wash Solutions and Fractions

Wash solutions compatible for use in the presently disclosed methods generally comprise one or more salts and optionally a buffer, such as a buffer described herein.

### First Wash Solutions

Methods of the present disclosure generally comprise a step of passing through or applying to an anion exchange medium a first wash solution, e.g., to obtain a first wash fraction, such as a wash fraction comprising empty viral capsid particles. Such wash solutions generally a quaternary ammonium salt. In many embodiments, (i) the concentration of the quaternary salt in the first wash solution remains constant throughout the step during which the first wash solution is passed through or applied to the anionic exchange medium; and/or (ii) the elution solution's salt composition remains constant throughout the step during which the elution solution is passed through or applied to the anionic exchange medium.

In some embodiments, the concentration of the quaternary ammonium salt in the wash solution is from 30 mM to 200 mM, including, e.g., from 50 mM to 180 mM, from 70 mM t 160 mM, from 90 mM to 150 mM, from 100 mM to 130 mM, or from 100 mM to 120 mM and all integers including and in between 30 mM to 200 mM.

In some embodiments, the concentration of the quaternary ammonium salt in the wash solution is at least 30 mM, at least 50 mM, at least 70 mM, at least 90 mM, or at least 100 mM.

In some embodiments, the concentration of the quaternary ammonium salt in the wash solution is no more than 200 mM, no more than 180 mM, no more than 160 mM, no more than 150 mM, no more than 140 mM, no more than 130 mM, or no more than 120 mM.

In some embodiments, the concentration of the quaternary ammonium salt in the wash solution is from 30 mM to 200 mM, including, e.g., from 50 mM to 180 mM, from 70 mM to 160 mM, from 90 mM to 150 mM, from 100 mM to 140 mM, from 100 mM to 130 mM, or from 100 mM to 120 mM and all integers including and in between 30 mM to 200 mM.

In some embodiments, the concentration of the quaternary ammonium salt in the wash solution is about 110 mM.

In some embodiments, the quaternary ammonium salt is a tetraalkylammonium salt, e.g., a tetraalkylammonium chloride or a tetraalkylammonium acetate. In some embodiments, the quaternary ammonium salt is a tetraalkylammonium chloride selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), or any combination(s) thereof. In some embodiments, the quaternary ammonium salt is tetraethylammonium chloride (TEAC).

In some embodiments, the quaternary ammonium salt is a tetraalkylammonium acetate selected from the group consisting of tetramethylammonium acetate, tetraethylammonium acetate (TEA-Ac), tetrapropylammonium acetate, tetrabutylammonium acetate, and any combination(s) thereof. In some embodiments, the quaternary ammonium salt is TEA-Ac.

In some embodiments, the quaternary ammonium salt is choline chloride.

In some embodiments, the first wash solution further comprises a divalent salt, e.g., MgCl₂, as further described herein. For example, in some embodiments, the first wash solution comprises MgCl₂ at a concentration from 1 mM to 10 mM, e.g., from 1 mM to 5 mM, including, e.g., about 2 mM and all integers in and between 1 mM to 10 mM.

In some embodiments, the first wash solution elutes empty capsid particles present in the viral capsid preparation, e.g., cause empty capsid particles that were present in the viral capsid preparation to come out in the first wash fraction. For example, in some embodiments, the first wash solution elutes 50% or more (e.g., from 50% to 100% including all integers in and between 50% to 100%), 60% or more (e.g., from 60% to 100%), 70% or more (e.g., from 70% to 100%), 75% or more (e.g., from 75% to 100), 80% or more (e.g., from 80% to 100%), 85% or more (e.g., from 85% to 100%), 90% or more (e.g., from 90% to 100%), 95% or more (e.g., from 95% to 100%), or 99% or more (e.g., from 99% to 100%) of the empty capsid particles present in the viral capsid preparation.

In some embodiments, the first wash fraction comprises 50% or more (e.g., from 50% to 100% including all integers in and between 50% to 100%), 60% or more (e.g., from 60% to 100%), 70% or more (e.g., from 70% to 100%), 75% or more (e.g., from 75% to 100%), 80% or more (e.g., from 80% to 100%), 85% or more (e.g., from 85% to 100%), 90% or more (e.g., from 90% to 100%), 95% or more (e.g., from 95% to 100%), or 99% or more (e.g., from 99% to 100%) of the empty capsid particles present in the viral capsid preparation.

In some embodiments, the first wash solution does not substantially elute full capsid particles present in the viral capsid preparation. For example, in some embodiments, the first wash solution elutes 50% or less (e.g., from 0 to 50% including all integers in and between 0 and 50%), 45% or less (e.g., from 0 to 40%), 40% or less (e.g., from 0 to 40%), 35% or less (e.g., from 0 to 35%), 30% or less (e.g., from 0 to 30%), 25% or less (e.g., from 0 to 25%), 20% or less (e.g., from 0 to 20%), 15% or less (e.g., from 0 to 15%), 10% or less (e.g., from 0 to 10%), 5% or less (e.g., from 0 to 5%), or 1% or less (e.g., from 0 to 1%) of the full capsid particles present in the viral capsid preparation.

In some embodiments, the first wash fraction comprises 50% or less (e.g., from 0 to 50% including all integers in and between 0 to 50%), 45% or less (e.g., from 0 to 45%), 40% or less (e.g., from 0 to 40%), 35% or less (e.g., from 0 to 35%), 30% or less (e.g., from 0 to 30%), 25% or less (e.g., from 0 to 25%), 20% or less (e.g., from 0 to 20%), 15% or less (e.g., from 0 to 15%), 10% or less (e.g., from 0 to 10%), 5% or less (e.g., from 0 to 5%), or 1% or less (e.g., from 0 to 1%) of the full capsid particles present in the viral capsid preparation.

### Second Wash Solutions

Methods of the present disclosure generally comprise a step of passing through or applying to an anion exchange medium a second wash solution, e.g., to obtain a second wash fraction, such as a wash fraction comprising the quaternary ammonium salt that was present in the first wash solution. In many embodiments, the second wash solution does not comprise a quaternary ammonium salt.

In some embodiments, the second wash solution comprises NaCl, Na₂SO₄, MgSO₄, or any combination thereof. For example, in some embodiments, the second wash solution comprises NaCl, e.g., at a concentration from 25 mM to 375 mM, from 50 mM to 250 mM, from 70 mM to 200 mM, from 70 mM to 140 mM, or from 90 mM to 140 mM, and including, all integers in and between 25 mM to 375 mM.

In some embodiments, the second wash solution elutes at least 30% (e.g., from 30% to 100% and including all integers in and between 30% to 100%), at least 40% (e.g., from 40% to 100%), at least 45% (e.g., from 45% to 100%), at least 50% (e.g., from 50% to 100%), at least 55% (e.g., from 55% to 100%), at least 60% (e.g., from 60% to 100%), at least 65% (e.g., from 65% to 100%), at least 70% (e.g., from 70% to 100%), at least 75% (e.g., from 75% to 100%), at least 80% (e.g., from 80% to 100%), at least 85% (e.g., from 85% to 100%), at least 90% (e.g., from 90% to 100%), at least 95% (e.g., from 95% to 100%), or at least 99% (e.g., from 99% to 100%), of the quaternary ammonium salt present in the first wash solution.

In some embodiments, the second wash fraction comprises at least 30% (e.g., from 30% to 100% and including all integers in and between 30% to 100%), at least 40% (e.g., from 40% to 100%), at least 45% (e.g., from 45% to 100%), at least 50% (e.g., from 50% to 100%), at least 55% (e.g., from 55% to 100%), at least 60% (e.g., from 60% to 100%), at least 65% (e.g., from 65% to 100%), at least 70% (e.g., from 70% to 100%), at least 75% (e.g., from 75% to 100%), at least 80% (e.g., from 85% to 100%), at least 85% (e.g., from 85% to 100%), at least 90% (e.g., from 90% to 100%), at least 95% (e.g., from 95% to 100%), or at least 99% (e.g., from 99% to 100%), of the quaternary ammonium salt present in the first wash solution.

In some embodiments, the second wash solution does not substantially elute full capsid particles present in the viral capsid preparation. For example, in some embodiments, the second wash solution elutes 30% or less (e.g., from 0 to 30% and including all integers in and between 0 to 30%), 25% or less (e.g., from 0 to 25%), 20% or less (e.g., from 0 to 30%), 15% or less (e.g., from 0 to 15%), 10% (e.g., from 0 to 10%), 8% or less (e.g., from 0 to 8%), 5% (e.g., from 0 to 5%), 3% or less (e.g., from 0 to 3%), 2% (e.g., from 0 to 2%), or 1% (e.g., from 0 to 1%) of the full capsid particles present in the viral capsid preparation.

In some embodiments, the second wash fraction comprises 30% or less (e.g., from 0 to 30% and including all integers in and between 0 to 30%), 25% or less (e.g., from 0 to 25%), 20% or less (e.g., from 0 to 20%), 15% or less (e.g., from 0 to 15%), 10% or less (e.g., from 0 to 10%), 8% or less (e.g., from 0 to 8%), 5% or less (e.g., from 0 to 5%), 3% or less (e.g., from 0 to 3%), 2% or less (e.g., from 0 to 2%), or 1% or less (e.g., from 0 to 1%) of the full capsid particles present in the viral capsid preparation.

### Elution Solutions and Fractions

Methods of the present disclosure generally comprise a step of passing through or applying to an anion exchange medium an elution solution, e.g., to elute full capsid particles in an elution fraction. In many embodiments, the elution solution does not comprise a quaternary ammonium salt.

In some embodiments, the elution solution comprises NaCl, Na₂SO₄, MgSO₄, or any combination thereof. For example, in some embodiments, the elution solution comprises NaCl, e.g., at a concentration from 25 mM to 375 mM, including, e.g., all integers in and between 25 mM to 375 mM, from 50 mM to 250 mM, from 70 mM to 200 mM, or from 70 mM to 140 mM, or 90 mM to 140 mM.

In some embodiments, the elution solution comprises an anionic species, tetrafluoroborate (BF₄), bromide (Br), or acetate (Ac). In some embodiments, the elution solution comprises an anionic species associated with tetraethylammonium (TEA), e.g., TEA-BF₄, TEA-Br, or TEA-Ac.

In some embodiments, the elution solution comprises at least one salt in common as the second wash solution, but at a different concentration. In some embodiments, the elution solution comprises a different salt than that present in the second wash solution.

In some embodiments, the elution solution substantially elutes the full capsid particles present in the viral capsid preparation. For example, in some embodiments, the elution solution elutes at least 15% (e.g., from 15% to 100% including all integers in and between 15% to 100%), at least 20% (e.g., from 20% to 100%), at least 25% (e.g., from 25% to 100%), at least 30% (e.g., from 30% to 100%), at least 35% (e.g., from 35% to 100%), at least 40% (e.g., from 40% to 100%), at least 45% (e.g., from 45% to 100%), at least 50% (e.g., from 50% to 100%), at least 55% (e.g., from 55% to 100%), at least 60% (e.g., from 60% to 100%), at least 65% (e.g., from 65% to 100%), at least 70% (e.g., from 70% to 100%), at least 75% (e.g., from 75% to 100%), at least 80% (e.g., from 80% to 100%), at least 85% (e.g., from 85% to 100%), at least 90% (e.g., from 90% to 100%), at least 95% (e.g., from 95% to 100%), or at least 99% (e.g., from 99% to 100%) of the full capsid particles present in the viral capsid preparation. In some embodiments, the elution solution elutes from 15% to 75% of the full capsid particles present in the viral capsid preparation.

In some embodiments, the elution fraction comprises at least 15% (e.g., from 15% to 100% including all integers in and between 15% to 100%), at least 20% (e.g., from 20% to 100%), at least 25% (e.g., from 25% to 100%), at least 30% (e.g., from 30% to 100%), at least 35% (e.g., from 35% to 100%), at least 40% (e.g., from 40% to 100%), at least 45% (e.g., from 45% to 100%), at least 50% (e.g., from 50% to 100%), at least 55% (e.g., from 55% to 100%), at least 60% (e.g., from 60% to 100%), at least 65% (e.g., from 65% to 100%), at least 70% (e.g., from 70% to 100%), at least 75% (e.g., from 75% to 100%), at least 80% (e.g., from 80% to 100%), at least 85% (e.g., from 85% to 100%), at least 90% (e.g., from 90% to 100%), at least 95% (e.g., from 95% to 100%), or at least 99% (e.g., from 99% to 100%) of the full capsid particles present in the viral capsid preparation. In some embodiments, the elution fraction comprises from 15% to 75% of the full capsid particles present in the viral capsid preparation.

In some embodiments, at least 35% (e.g., from 35% to 100% including all integers in and between 35% to 100%), at least 40% (e.g., from 40% to 100%), at least 45% (e.g., from 45% to 100%), or at least 50% (e.g., from 50% to 100%) of the capsid particles present in the elution fraction are full capsid particles. In some embodiments, at least 50% (e.g., from 50% to 100%) of the capsid particles present in the elution fraction are full capsid particles.

In some embodiments, the elution fraction comprises no more than 65% (e.g., from 0 to 65% including all integers in and between 0 to 65%), no more than 60% (e.g., from 0 to 60%), no more than 55% (e.g., from 0 to 55%), no more than 50% (e.g., from 0 to 50%), no more than 45% (e.g., from 0 to 45%), no more than 40% (e.g., from 0 to 40%), no more than 35% (e.g., from 0 to 35%), no more than 30% (e.g., from 0 to 30%), no more than 25% (e.g., from 0 to 25%), no more than 20% (e.g., from 0 to 20%), no more than 15% (e.g., from 0 to 15)%, no more than 10% (e.g., from 0 to 10%), no more than 5% (e.g., from 0 to 5%), or no more than 1% (e.g., from 0 to 1%) of the empty capsid particles from the viral capsid preparation.

### Buffer Systems and Other Solution Components

In some embodiments, one or more of the first wash solution, the second wash solution, and the elution solution comprise a buffer system. For example, the buffer system may maintain the pH around a certain value or within a certain range, a pH from 6.0 to 10.0, e.g., from 7.0 to 9.0. In some embodiments, an included buffer maintains the pH of a solution at about 6.0, about 6.5, about 7.0, about 7.5, about 8.0, about 8.5, about 9.0, about 9.5, or about 10.0. In some embodiments, an included buffer maintains the pH of a solution at about pH 9.0.

Non-limiting examples of suitable buffer systems include bis-Tris Propane-based buffers, such as a 20 mM bis-Tris Propane system.

In some embodiments, one or more of the first wash solution, the second wash solution, and the elution solution comprises a divalent salt, e.g., MgCl₂. In some embodiments, one or more the first wash solution, the second wash solution, and the elution solution comprise(s) MgCl₂ at a concentration from 0.5 mM to 10 mM, e.g., from 1 mM to 10 mM, or from 1 mM to 5 mM, such as about 1 mM, about 2 mM, about 3 mM, about 4 mM, about 5 mM, about 6 mM, about 7 mM, about 8 mM, about 9 mM, or about 10 mM. In some embodiments, the one or more the first wash solution, the second wash solution, and the elution solution comprises MgCl₂ at a concentration of about 2 mM. In some embodiments, the one or more the first wash solution, the second wash solution, and the elution solution comprises MgCl₂ at a concentration of at least 1 mM or at least 2 mM. In some embodiments, one or more the first wash solution, the second wash solution, and the elution solution comprises MgCl₂ at a concentration no greater than 5 mM.

In some embodiments, one or more of the first wash solution, the second wash solution, and the elution solution comprises a stabilizer or a surfactant, e.g., a non-ionic surfactant. Non-limiting examples of suitable non-ionic surfactants include, e.g., Pluronic® F-68. In some embodiments, the stabilizer or surfactant is present in a solution at a concentration of about 0.0001%, about 0.0005%, or about 0.001%. In some embodiments, the stabilizer or surfactant is present in a solution at a concentration of no greater than about 0.001%.

### Separation Methods

Methods of the present disclosure typically include a wash step using a wash solution comprising a quaternary ammonium salt, wherein the concentration of the quaternary ammonium salt in that wash solution remains constant (that is, is held isocratically) throughout the wash step. In some embodiments, the concentrations of all salts in the wash solution comprising the quaternary ammonium salt are held isocratically throughout the wash step. In some embodiments, the concentrations of all components in the wash solution comprising the quaternary ammonium salt are held isocratically throughout the wash step.

Other steps of the presently disclosed methods, e.g., additional wash steps or elution steps, may involve isocratic separation or gradient separation, as described further herein.

### Isocratic Separation

As mentioned, in many embodiments, (i) the concentration of the quaternary salt in the first wash solution remains constant throughout the step during which the first wash solution is passed through or applied to the anion exchange medium and/or (ii) the elution solution's salt composition remains constant throughout the step during which the elution solution is passed through or applied to the anion exchange medium. In some embodiments, concentrations of additional components of the first wash solution also remain constant throughout the step. For example, in some embodiments, the first wash solution's salt composition remains constant throughout the step.

In some embodiments, one or both of (1) the step of passing through or applying the second wash solution to the anion exchange medium and (2) the step of passing through or applying the elution solution to the anion exchange medium involves an isocratic separation. In some embodiments, the salt composition of the second wash solution and/or the salt composition of the elution solution remains constant throughout the steps during which the solutions are passed through or applied to the anion exchange medium.

In some embodiments, first wash solution's salt composition, the second wash solution's salt composition, and the elution solution's salt composition remain constant throughout each individual wash or elution step.

### Gradient Separation

In certain embodiments, one or more steps (e.g., a wash step and/or an elution step) in a method disclosed herein comprises using a solution whose composition varies during that step or those steps. For example, in a gradient separation step, the concentration of a salt in the solution may gradually and continually increase (e.g., linearly) over time throughout the step.

Assessment of Viral Capsid Preparations and/or Fractions

In certain embodiments, the viral capsid preparation and/or the elution fraction, or a sample thereof, is assessed. The first and second wash fractions may or may not be collected. In some embodiments, at least a sample of one or both wash fractions are collected and assessed, e.g., for quality control purposes.

### Full and Empty Capsid Particles

In some embodiments, the presence and/or amount(s) of full and/or empty capsid particles in the viral capsid preparation and/or one or more fractions (e.g., an elution fraction) is assessed. Various methods are known in the art to determine the presence of full or empty capsids; many of these methods can also be used to determine amounts, e.g., relative amounts of full and empty capsids. Examples of such methods include, but are not limited to, transmission electron microscopy (TEM), sedimentation velocity-analytical ultracentrifugation (SV-AUC), charge detection mass spectrometry (CDMS), anion exchange high performance liquid chromatography (AEX-HPLC), UV spectrophotometry, and measuring capsid and genome copies by ELISA and qPCR, e.g., for quality control purposes.

In some embodiments, an ultracentrifugation method is used to assess a viral capsid preparation and/or a fraction (e.g., an elution fraction). For example, SV-AUC is a solution-state method that measures the rate of sedimentation of molecules when subject to high spinning speed that applies a centrifugal force. The sedimentation rate, measured by sedimentation coefficient s, is related to the buoyant mass, density, specific volume, and the friction force of the molecule in its formulation matrix. The s-value, when normalized to standard solution conditions of water at 20° C. (standard temperature and pressure) is known as the s₂₀, w value, a fundamental molecular parameter that defines the mass and shape as well as the conformation of the molecule. The sedimentation coefficient distribution can be quantitated by area under peak, which is directly related to the quantity of molecules at that sedimentation coefficient. The degree of accuracy of the peak area in representing the true population depends partially on the suitability of detection system, and the number of data points gathered.

SV-AUC may be applied, for example, to separate different types of capsid particles, e.g., full from empty capsid particles. The masses of virus particles having the same virus type and serotype may differ depending on the presence of a complete vector genome (as with full capsid particles), or presence of only part of a vector genome or complete absence of a vector genome (as with empty capsid particles). For example, empty capsid particles, having less DNA than full capsids, would be lighter than full capsids and would sediment more slowly than would full capsids. Thus, the s-value in an SV-AUC method would reflect the size of DNA packaged within a viral capsid particle.

In some embodiments, a UV spectrophotometry method is used to assess a viral capsid preparation and/or a fraction (e.g., an elution fraction). For example, the amount of light at wavelengths at or around 254 nm and/or around 260 nm that is absorbed by sample is generally proportional to the concentration of nucleic acids in the sample. Additionally, proteins have a greater absorbance at 280 nm (A₂₈₀) than they do at 254 nm (A₂₅₄) or 260 nm (A₂₆₀); the inverse is true for nucleic acids, which have a greater A₂₅₄ or A₂₆₀ than A₂₈₀. Thus, full capsid particles, which have a greater amount of DNA than do empty capsid particles, will have a greater A₂₅₄/A₂₈₀ or A₂₆₀/A₂₈₀ ratio than would empty capsid particles. This difference can be exploited to assess the relative amounts of full and empty particles in a sample. In some embodiments, one or more of A₂₅₄, A₂₆₀, A₂₈₀, A₂₅₄/A₂₈₀ ratio, or A₂₆₀/A₂₈₀ ratio, is assessed.

Additionally, some impurities in a sample may absorb light at a wavelength of 230 nm; these contaminants are typically more numerous than those contaminants that absorbed at 280 nm. Thus, the A₂₆₀/A₂₃₀ ratio may provide some indication of purity of a sample. In some embodiments, the A₂₃₀ and/or A₂₆₀/A₂₃₀ value is assessed.

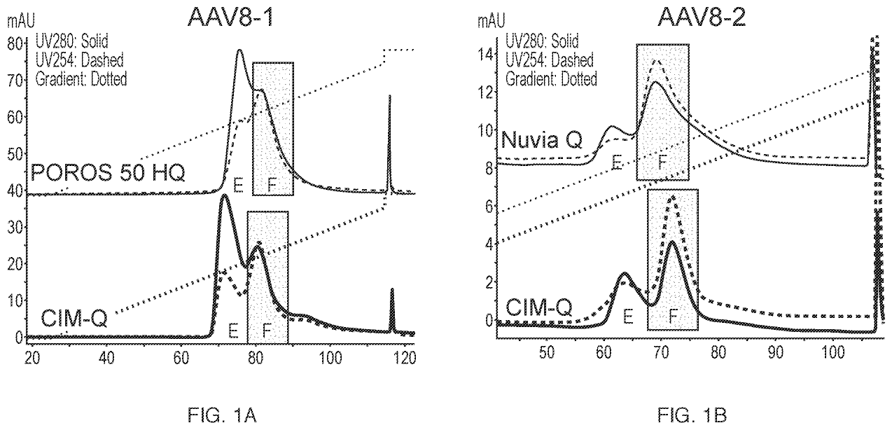

For example, in some embodiments, empty capsid particles are preferentially released from the AEX medium before full capsid particles are released. In these embodiments, the A₂₅₄/A₂₈₀ or A₂₆₀/A₂₈₀ ratio of one fraction (in which empty capsid particles are preferentially released) will be less than that of the subsequent fraction (in which full capsid particles are preferentially released) (e.g., as shown in FIG. 1).

In some embodiments, full capsid particles are preferentially released from the AEX medium before empty capsid particles are released. In these embodiments, the A₂₅₄/A₂₈₀ ratio or A₂₆₀/A₂₈₀ ratio of one fraction (in which full capsid particles are preferentially released) will be greater than that of a subsequent fraction (in which empty capsid particles are preferentially released).

The ordering of preferential release may be associated with one or more of various aspects such as, for example (but not limited to) a) the characteristics of the nucleic acid payload, b) capsid serotype, c) viral capsid (e.g., rAAV) preparation conditions, and d) characteristics of the AEX medium.

### Other Assessments

In some embodiments, a fraction or a sample thereof is assessed to determine the presence or amount of an analyte, e.g., a component of a wash solution. For example, in some embodiments, a sample of the second wash fraction is assessed to determine the presence or amount of quaternary ammonium salt in the sample. Methods of detecting or quantitating quaternary ammonium salts are known in the art and include, e.g., liquid chromatography-mass spectrometry (LC-MS) and reversed-phase high-performance liquid chromatography (RP-HPLC).

### EXAMPLES

### Example 1: Impact of Stationary Phase on Separation of Empty and Full Capsid Particles in an NaCl Gradient Method

To test the impact of types of stationary phases on separation of empty and full capsid particles, viral capsid preparations comprising full and empty AAV capsid particles were subjected to NaCl gradient elution conditions on different types of stationary phases.

Both monolithic and bead-based anion exchange media were tested. CIMmultus® QA columns (BIA Separation) are monolithic poly(glycidyl methacrylate-co-ethylene dimethacrylate) columns functionalized with quaternary amines, strong anion exchangers. POROS™ 50 HQ columns (Thermo Scientific) are based on poly(styrene-divinylbenzene) beads functionalized with quaternized polyetheleimine, strong anion exchangers. Nuvia™ Q (Bio-Rad) columns are based on poly(methacrylate) beads functionalized with quaternary amines, strong anion exchangers.

AAV8.1 material comprising empty and full capsid particles was subjected to an NaCl gradient on POROS™ 50 HQ columns, and, in a separate experiment, on CIMmultus® QA columns. AAV8.2 material comprising empty and full capsid particles was subjected to an NaCl gradient on Nuvia™ Q columns, and, in a separate experiment, on CIMmultus® QA columns.

FIG. 1A shows representative chromatograms from the separation experiments on AAV8.1 material.

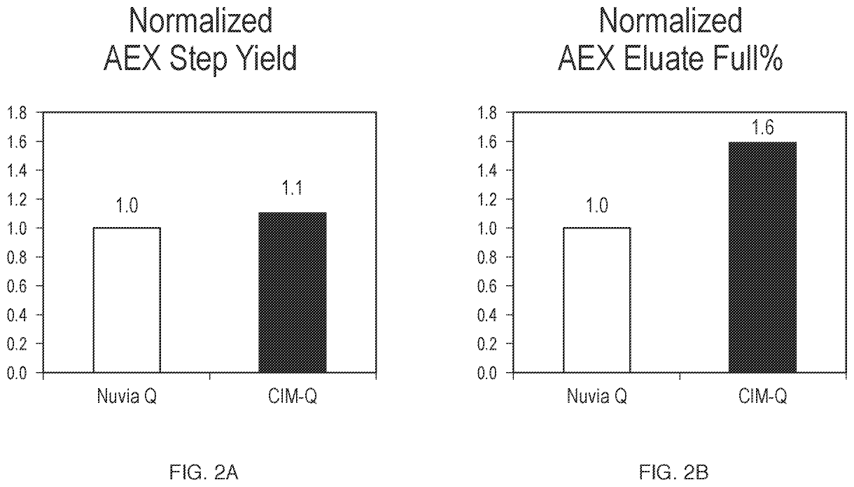

FIG. 1B shows representative chromatograms from the separation experiments on AAV8.2 material. FIG. 2A depicts the step yield, the amount of genome of interest in the pooled fraction relative to the amount in load material, as determined by quantitative polymerase chain reaction (qPCR) methods. FIG. 2B depicts the percentage of full capsid particles present in eluates, as determined by analytical ultra-centrifugation (AUC). Values in both FIGS. 2A and 2B are normalized to those observed with the Nuvia™ Q columns.

For AAV8-1, using CIMmultus® Q columns resulted in better separation of empty capsids (high amounts are found in fractions denoted by “E”) from full capsids (high amounts are found in fractions denoted by “F”) than using POROS™ 50 HQ columns. For AAV8-2, using CIMmultus® QA columns resulted in better separation of empty capsids (high amounts are found in fractions denoted by “E”) from full capsids (high amounts are found in fractions denoted by “F”) than using Nuvia™ Q columns. Moreover, for AAV8-2, both the step yields and the percentages of full capsid particles were higher in experiments using the CIMmultus® Q columns.

### Example 2: Impact of Producer Cell Line and AAV Serotype on Separation of Empty and Full Capsid Particles

To test the impact of the load material (producer cell line and AAV serotype) on separation of empty and full capsid particles, various viral capsid preparations comprising full and empty viral particles were subjected to NaCl gradient elution conditions on CIMmultus® QA columns.

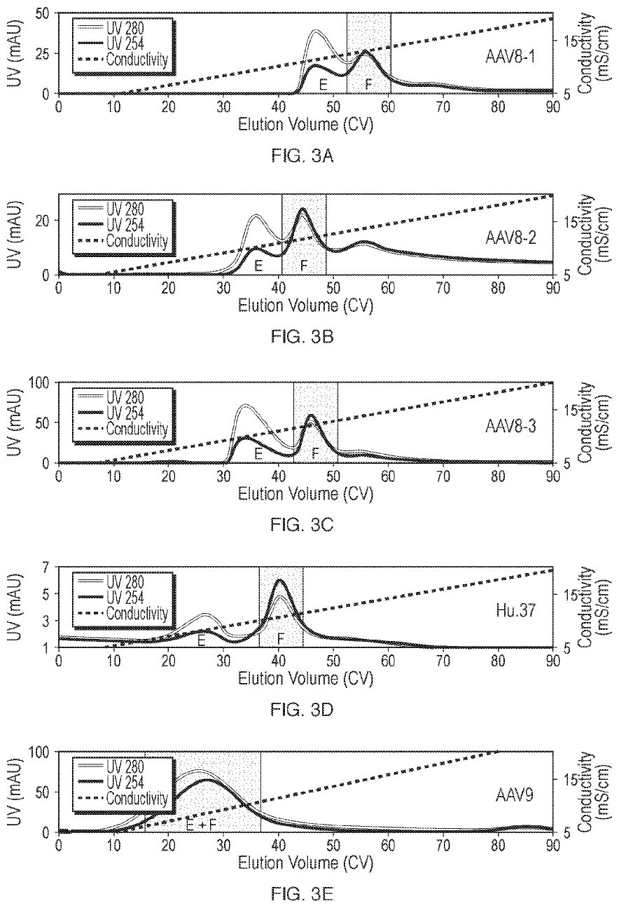

HEK-produced AAV of serotypes AAV8-1, AAV8-2, AAV8-3, and hu37 and HeLa-produced AAV9 were subjected to NaCl gradient elution conditions. The results are shown in FIG. 3 and in Table 1. FIG. 3 depicts representative chromatograms from experiments using the various AAV materials. “E” denotes fractions corresponding to high relative amounts of empty capsids, and “F” denotes fractions corresponding to high relative amounts of full capsids.

TABLE 1
Resolution between peaks for AAV material of various
serotypes and produced by HEK or HeLa cell lines

| Cell line | Serotype | Resolution (between “E” peak and “F” peak) |
|---|---|---|
| HEK | AAV8-1 | 0.75 |
| HEK | AAV8-2 | 0.68 |
| HEK | AAV8-3 | 1.14 |
| HEK | hu37 | 1.30 |
| HeLa | AAV9 | N/A |

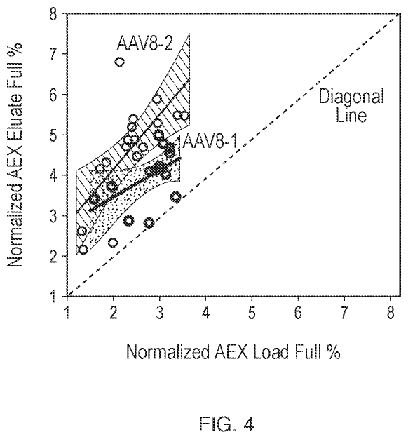

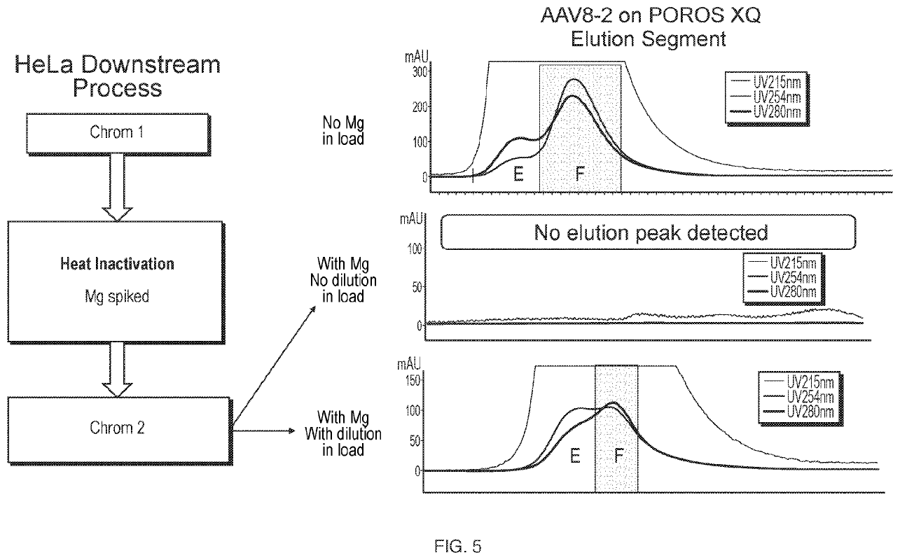

FIG. 4 depicts graphs comparing results from experiments run on HEK-produced AAV8-1 and AAV8-2, showing the normalized percentages of full capsid particles in eluate (y-axis) as compared to percentages of full capsid particles in the load material (x-axis).

### Example 3: Impact of Load Salt Concentration on Separation of Empty and Full Capsid Particles

To test the impact of load salt concentration on separation of empty and full capsid particles, viral capsid preparations comprising full and empty HeLa-produced AAV8.2 capsid particles were subjected to a downstream process involving variable amounts of MgCl₂.

After an initial chromatography process, AAV material produced from HeLa cells was heat-inactivated to inactivate any helper viruses, and samples were spiked with 200 mM MgCl₂ to a final concentration of either 67 mM (“no dilution in load”) or 6-7 mM (“dilution in load”). Another sample was not spiked with any MgCl₂ (“no Mg in load”). The left-hand side of FIG. 5 shows an outline of the process.

Samples were then subject to NaCl gradient elution conditions on POROS™ XQ columns. Representative chromatograms from this experiment are depicted on the right-hand side of FIG. 5. In FIG. 5, “E” denotes fractions corresponding to high relative amounts of empty capsids, and “F” denotes fractions corresponding to high relative amounts of full capsids.

As shown in FIG. 5, no elution peak was detected for the “no dilution in load” (67 mM MgCl₂) sample, whereas an elution peak was detection when MgCl₂ concentration was diluted down to 6-7 mM.

### Example 4: Gradient Elution with Varying Tetramethylammonium Chloride (TMAC)

In this example, viral capsid preparations comprising full and empty viral capsid particles were subjected to gradient elution conditions using varying tetramethylammonium chloride (TMAC) concentrations. As a comparison, a similar elution experiment was conducted using NaCl. Concentrations of TMAC or of NaCl were increased over time.

### Materials and Methods

The AAV material was obtained from a pilot scale 250 L bioreactor operation. The clarified harvest was affinity captured and affinity eluate samples were retained for lab scale AEX runs. 1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (NaCl buffer: 20 mM BTP, 25 mM NaCl, 0.001% (w/v) Pluronic® F-68, pH 9.0; TMAC buffer: 20 mM BTP, 25 mM TMAC, 0.001% (w/v) Pluronic® F-68, pH 9.0). After washing with 10 column volumes (CV) of equilibration buffer, a linear gradient from about 25 mM to about 182.5 mM NaCl and about 25 mM to about 326.7 mM TMAC, generated over 90 CVs was applied for the NaCl and TMAC case, respectively. The eluate was collected in fractions and fractions corresponding to “Empty” and “Full” peaks were pooled accordingly.

The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

Relative amounts of full versus empty viral capsid particles that eluted in each peak were assessed by determining absorbances at 254 nm (A₂₅₄) and at 280 nm (A₂₈₀).

### Results and Discussion

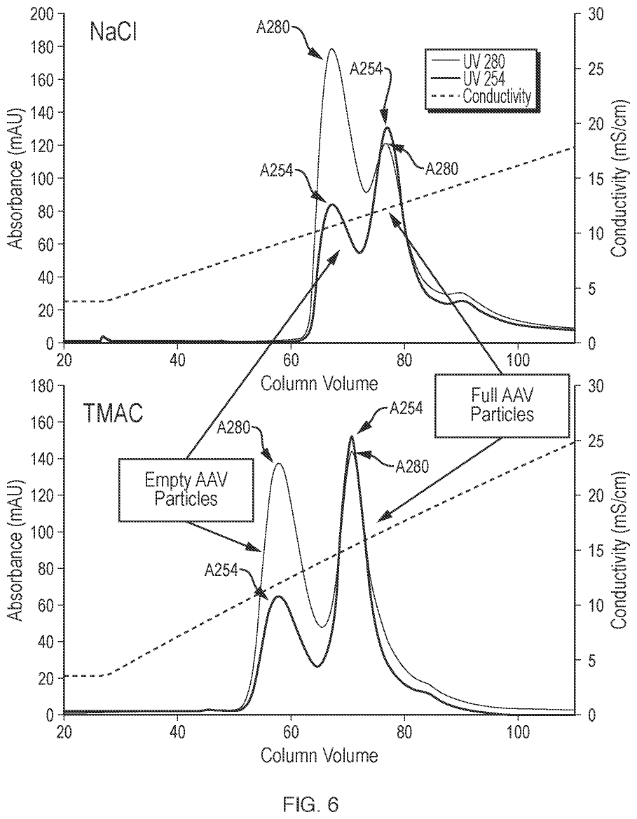

FIG. 6 depicts exemplary chromatograms from these experiments.

The chromatograms of FIG. 1 depict A₂₅₄ and A₂₈₀ measurements of eluate during gradient elution. As shown, a first fraction is enriched in “empty” AAV particles (A₂₅₄/A₂₈₀ ratio of the first elution peaks are usually no more than one) while a second, later eluting fraction is enriched in “full” AAV particles. (The A₂₅₄/A₂₈₀ ratio of the second elution peaks are usually greater than one). The rising straight line shows the increase in solution conductivity caused by the corresponding increase in eluent concentration.

The present Example demonstrates that a gradient elution method using TMAC achieves greater separation of empty and full capsids than a comparable gradient elution method using NaCl.

### Example 5: Gradient Elution with Varying Tetramethylammonium Chloride (TMAC), Tetrabutylammonium Chloride (TBAC), or Benzyltributylammonium Chloride (BTBAC) and Constant MgCl₂ Concentration

In this example, viral capsid preparations comprising full and empty viral capsid particles were subjected to gradient elution conditions using a series of tetraalkylammonium salts, and the yield of full viral capsid particles was assessed.

### Materials and Methods

AAV material was obtained similarly as described in Example 1. 1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media (in this case, a CIMmultus® QA column) pre-equilibrated with pH-matched equilibration buffer (20 mM BTP, 25 mM NaCl, 2 mM MgCl₂, pH 9.0). For elution with NaCl, after washing with 10 column volumes (CV) of equilibration buffer, a linear gradient from about 25 mM to about 182.5 mM NaCl, generated over 90 CVs was applied. For elution with a QA salt (TMAC, TBAC, or BTBAC), after washing with 10 column volumes (CV) of equilibration buffer, a linear gradient from about 25 mM to about 337.5 mM QA, generated over 90 CVs was applied. The eluate was collected in fractions and fractions corresponding to “Empty” and “Full” peaks were pooled accordingly. The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

### Results and Discussion

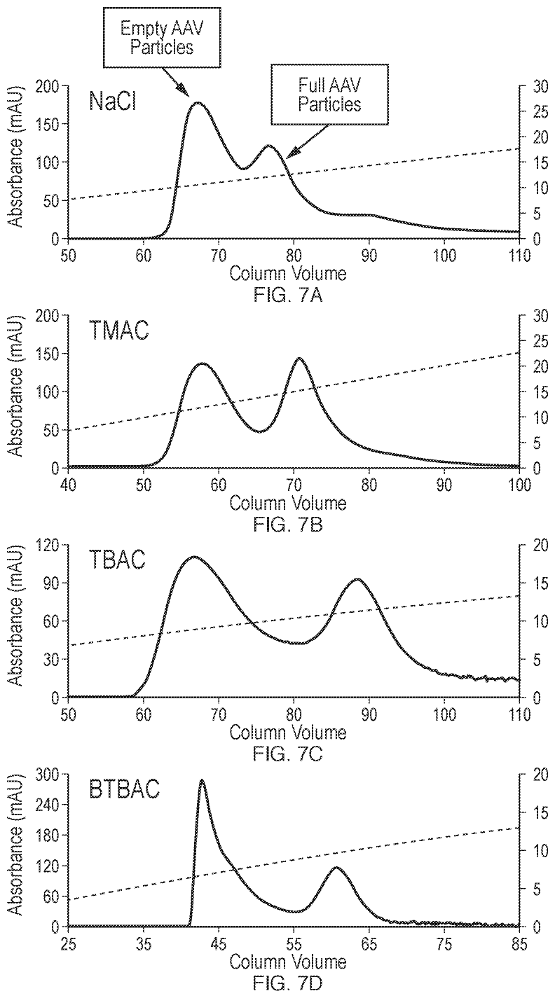

FIGS. 7A-7D depict exemplary chromatograms for empty-AAV particle to full-AAV particle separations using gradient elution with NaCl or different QAs with increasing the size of the functional groups. From top to bottom, the elution was obtained from a sodium chloride (NaCl) (FIG. 7A), tetramethylammonium chloride (TMAC) (FIG. 7B), tetrabutylammonium chloride (TBAC) (FIG. 7C), or benzyltributylammonium chloride (BTBAC) (FIG. 7D) gradient. A chromatogram of a NaCl gradient is depicted as a reference for comparing empty and full fraction resolution. The chromatograms depict A₂₅₄ and A₂₈₀ measurements of eluate during a gradient elution. The rising straight line shows the increase in solution conductivity caused by the corresponding increase in eluent concentration.

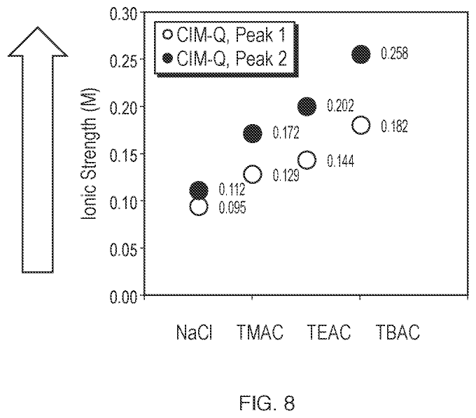

FIG. 8 depicts a graph plotting ionic strength (x-axis) of the first and second peaks as a function of the size of positive ions. As can be seen in FIGS. 7 and 8, increasing differences between the peaks are observed with increasing size of positive ions.

This example demonstrates the increased separation of full and empty viral capsid particles as the size of the cation (Na or QA) added to the eluent increases.

### Example 6: Gradient Elution with Varying Tetraethylammonium Chloride (TEAC) and Constant MgCl₂ Concentration

In this example, viral capsid preparations comprising empty and full-AAV particles were separated using a gradient elution with a tetraethylammonium chloride (TEAC) gradient on a CIMmultus® QA column with a constant magnesium chloride (MgCl₂) concentration of 2 mM at pH 9.

### Materials and Methods

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM TEAC, 2 mM MgCl₂, pH 9.0). After washing with 10 column volumes (CV) of equilibration buffer, a linear gradient from about 25 to about 337.5 mM TEAC (the concentration of MgCl₂ is kept constant at 2 mM), generated over 90 CVs, was applied. The eluate was collected in fractions and fractions corresponding to “empty” and “full” peaks were pooled accordingly. The column was then washed with 10 CV of a high-salt strip solution (20 mM BTP, 2 M NaCl, 2 mM MgCl₂, pH 9.0), followed by 10 CV of a sanitation solution (3 M NaCl, 1 M NaOH), and subsequently 10 CV of a column-storage solution (50 mM Tris, 150 mM NaCl, 20% ethanol, pH 7.5).

### Results and Discussion

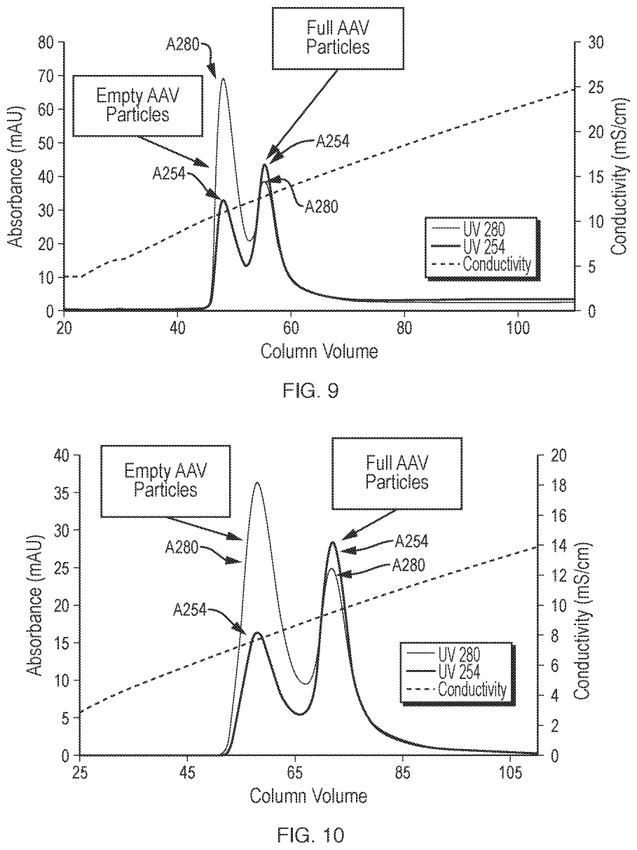

FIG. 9 depicts chromatograms from this experiment. The chromatograms depict A₂₅₄ and A₂₈₀ measurements of eluate during a gradient elution. The rising straight line shows the increase in solution conductivity caused by the corresponding increase in TEAC concentration.

This example demonstrates the separation of empty and full AAV particles using a TEAC gradient elution.

### Example 7: Gradient Elution with Varying Tetraethylammonium Acetate (TEA-Ac) and Constant MgCl₂ Concentration

In this example, viral capsid preparations comprising full and empty viral capsid particles were subjected to gradient elution conditions using tetraethylammonium acetate (TEA-Ac). The concentration of TEA-Ac increased over time, and the MgCl₂ concentration remained constant at 2 mM MgCl₂.

### Materials and Methods

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM NaCl, 2 mM MgCl₂, pH 9.0). After washing with 10 column volumes (CV) of equilibration buffer, a linear gradient from about 25 mM to about 337.5 mM TEA-Ac, generated over 90 CVs, was applied. The eluate was collected in fractions and fractions corresponding to “empty” and “full” peaks were pooled accordingly. The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

### Results and Discussion

FIG. 10 depicts chromatograms from this experiment. The chromatograms depict A₂₅₄ and A₂₈₀ measurements of eluate during a gradient elution. The rising straight line shows the increase in solution conductivity caused by the corresponding increase in TEA-Ac concentration.

This Example demonstrates separation of full and empty AAV particles from a viral capsid preparation, using a TEA-Ac gradient.

### Example 8: Isocratic Elution with Tetraethylammonium Acetate (TEAC) Using a Wash Solution, Secondary Wash Solutions, and an Elution Solution

In this example, viral capsid preparations comprising full and empty viral capsid particles were subjected to an isocratic elution involving an initial wash with tetraethylammonium chloride (TEAC) and magnesium chloride (MgCl₂), followed by two sequential step washes with sodium chloride (NaCl) and MgCl₂, and followed by elution with NaCl and MgCl₂.

### Materials and Methods

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM NaCl, 2 mM MgCl₂, pH 9.0). After washing with 10 column volumes (CV) of equilibration buffer, a first 10 CVs isocratic wash with TEAC (20 mM BTP, 124.75 mM TEAC, 2 mM MgCl₂, pH 9.0) was applied to wash out empty capsid from AEX media. After the first wash, a second isocratic wash containing only NaCl (20 mM BTP, 68.75 mM NaCl, 2 mM MgCl₂, pH 9.0) was applied to remove residual TEAC salt from AEX media. Finally, the AAV particles (full capsid enriched) were eluted from AEX media by elution buffer (20 mM BTP, 109.35 mM NaCl, 2 mM MgCl₂, pH 9.0) and collected. The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

### Results and Discussion

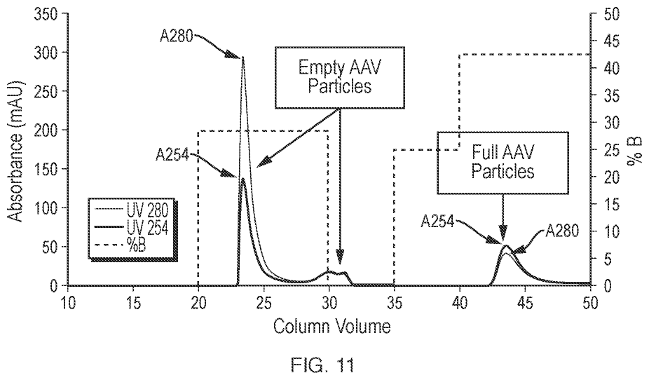

FIG. 11 depicts a chromatogram from these experiments. The peaks corresponding the full and empty AAV particles are indicated in FIG. 11 and are clearly separated. The dashed line corresponds to the volumetric output of pump “B” (i.e., the pump(s) that feed in wash and elution buffers) relative to pump “A” (i.e., the pump(s) that feed in equilibration buffers).

This example demonstrates the successful separation of empty and full viral capsid particles using isocratic elution with TEAC.

### Example 9: Comparison of a Variety of Gradient and Isocratic Protocols on Separation of Full and Empty Capsid Particles

To compare various gradient and isocratic elution protocols for their ability to separate full and empty particles, AAV8-1 material comprising full and empty capsid particles were subjected to various protocols using MgSO₄, and all on a CIMmultus® QA column.

The following protocols were tested: 1) a gradient elution; 2) a gradient wash followed by an isocratic elution step; 3) a single isocratic wash; 4) three isocratic washes followed by an isocratic elution; and 5) two isocratic washes followed by an isocratic elution.

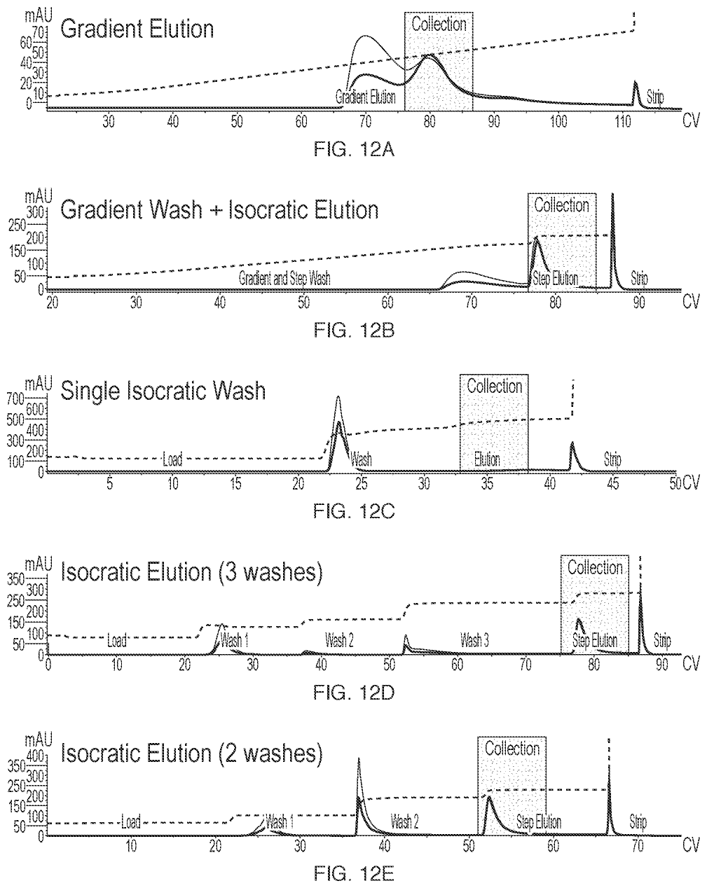

FIGS. 12A-12E depict exemplary chromatograms from these experiments. As shown in FIGS. 12A-12E, the isocratic elution strategies resulted in better percentages of full capsid particles collected and better step yield (of the genome of interest), than did the gradient elution method.

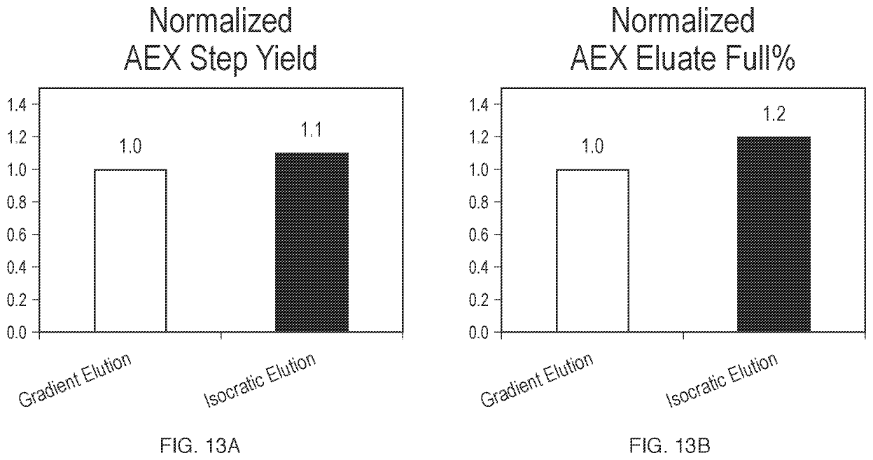

FIGS. 13A and 13B depict graphs comparing results from gradient elution protocols compared to results from isocratic elution protocols. FIG. 13A depicts the step yield, the amount of genome of interest in the pooled fraction relative to the amount in load material, as determined by quantitative polymerase chain reaction (qPCR) methods. FIG. 13B depicts the percentage of full capsid particles present in eluates, as determined by analytical ultra-centrifugation (AUC). Values in both FIGS. 13A and 13B are normalized to those observed with the gradient elution protocols.

### Example 10: Gradient Elution with Varying Choline Chloride Concentrations and Constant MgCl₂ Concentration

In this example, viral capsid preparations comprising full and empty viral capsid particles were subjected to gradient elution conditions using choline chloride. The concentration of choline chloride increased over time, and the MgCl₂ concentration remained constant at 2 mM MgCl₂.

### Materials and Methods

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM NaCl, 2 mM MgCl₂, pH 9.0). After washing with 10 column volumes (CV) of equilibration buffer, a linear gradient from about 25 mM to about 340 mM choline chloride, generated over 90 CVs was applied. The eluate was collected in fractions and fractions corresponding to “empty” and “full” peaks were pooled accordingly. The column was then washed with 10 CV of a high-salt strip solution (20 mM BTP, 2 M NaCl, 2 mM MgCl₂, 25 pH 9.0), followed by 10 CV of a sanitation solution (3 M NaCl, 1 M NaOH), and subsequently 10 CV of a column-storage solution (50 mM Tris, 150 mM NaCl, 20% ethanol, pH 7.5).

### Results

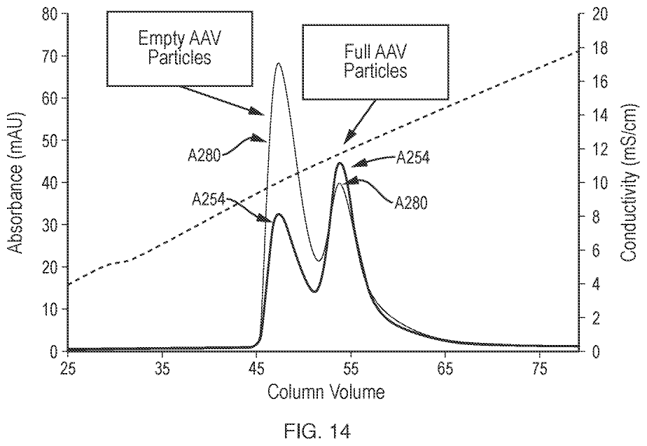

FIG. 14 depicts an exemplary chromatogram from this experiment. As shown in FIG. 14, gradient separation using choline chloride results in separation of full and empty AAV capsids from the viral capsid preparation.

### Example 11: Optimization with Respect to Host Cell Proteins and Elution of Full Capsids

To test the effects of column loading and load residence time on the amounts of host cell protein and of full capsid particles in eluates, different run conditions were tested with AAV material comprising full and empty capsid particles.

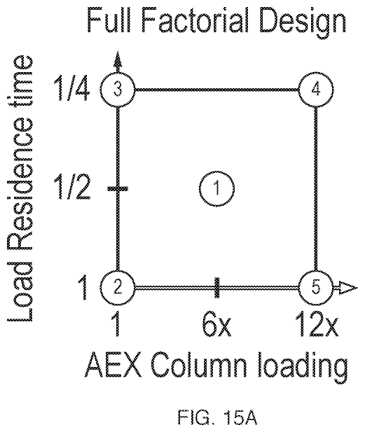

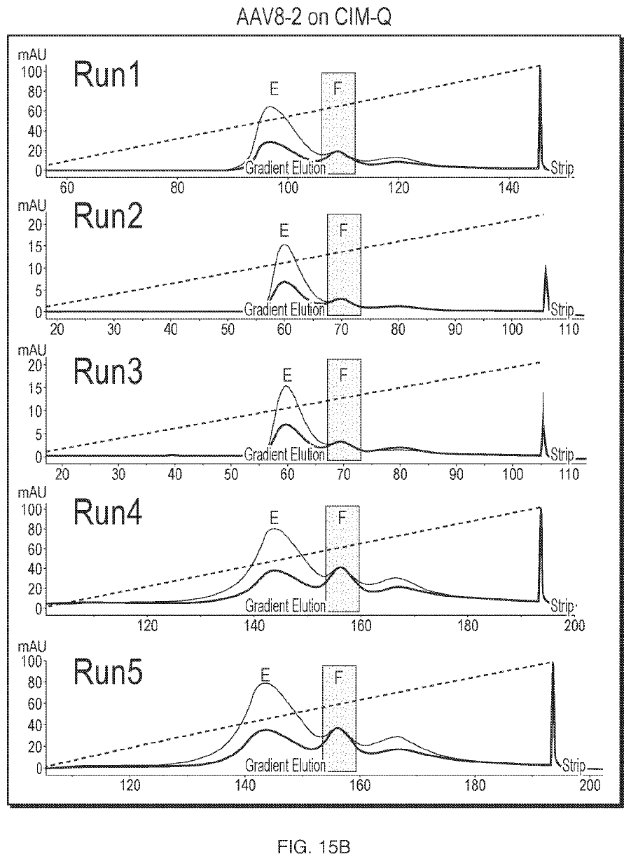

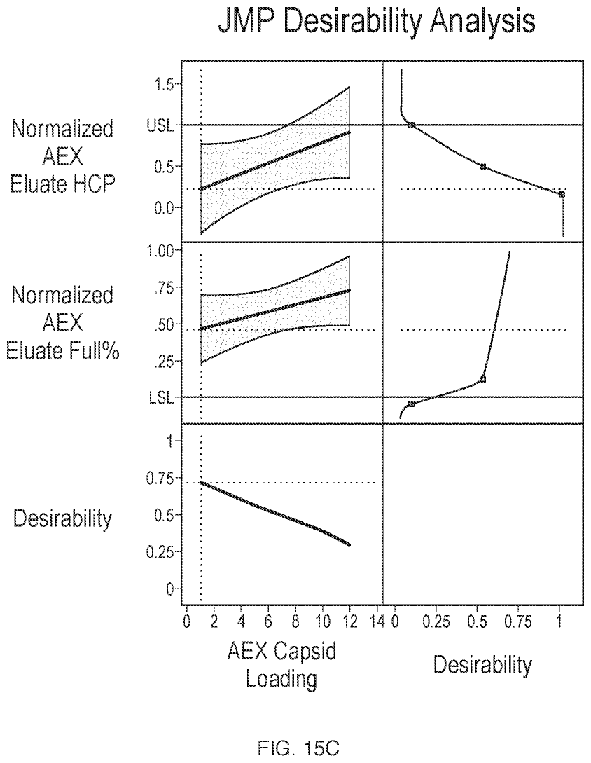

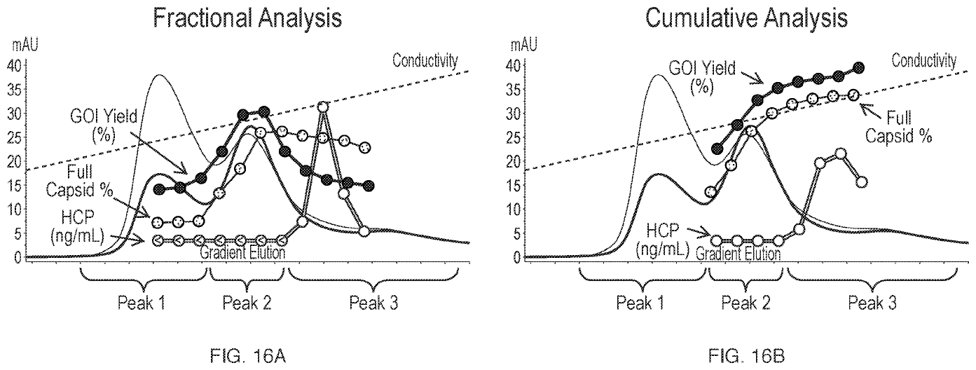

FIG. 15A depicts the parameters for the five different runs tested in this set of experiments. Columns were loaded with 1×, 6×, and 12× amount of AAV8-2 material, with a residence time of 1×, ½×, or ¼×. Samples were run on CIMmultus® QA columns using an NaCl gradient. FIG. 15B depicts chromatograms from the various runs whose conditions are shown in FIG. 15A. “E” denotes fractions corresponding to high relative amounts of empty capsids, and “F” denotes fractions corresponding to high relative amounts of full capsids.

FIG. 15C shows plots from a desirability analysis using JMP® software, for variables such as capsid loading, normalized host cell protein amounts in eluate, and normalized percentages of full capsids in eluates.

As shown in FIG. 15C, increased capsid loading led to increased percentages of full capsids in eluates, but also increased amounts of host cell protein in eluates.

FIGS. 16A and 16B show the yields (of genome of interest), percentages of full capsids, and host cell protein amounts from similar experiments using AAV8-1 material run on CIMmultus® QA columns using an NaCl gradient. FIG. 16A shows amounts by fraction, and FIG. 16B shows cumulative amounts. As can be seen in FIGS. 16A and 16B, fractions after the second peak have detectable host cell protein levels, and the collecting additional fractions to increase the percentage of full capsid proteins collected also increases the cumulative amounts of host cell protein.

### Example 12: Use of Divalent Ions in the Mobile Phase in Separating Empty and Full Capsid Particles

To test the effect of using a mobile phase with divalent ions on the separation of full and empty capsid particles, samples of AAV8-1 and AAV8-2 material comprising full and empty capsid particles were run on CIMmultus® QA columns using either an NaCl gradient or a gradient with a mobile phase comprising MgSO₄.

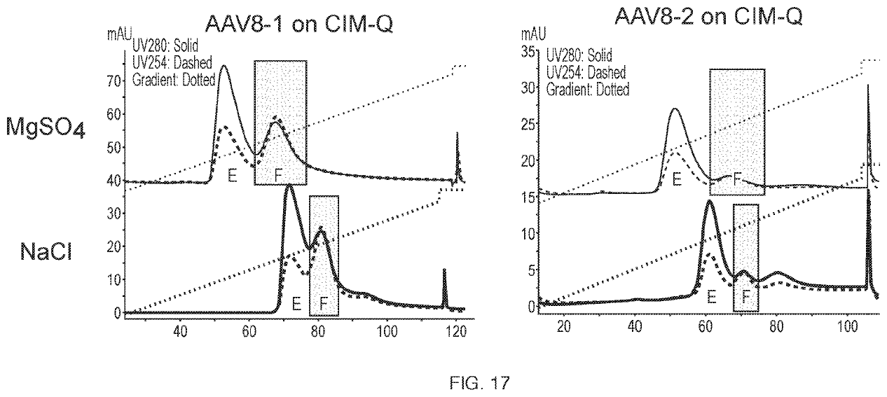

FIG. 17 depicts representative chromatograms from these experiments. As shown in FIG. 17, using a mobile phase comprising divalent ions enhanced the separation of full and empty capsid particles for both AAV8-1 and AAV8-2.

### Example 13: Integrated Optimization of the Mobile Phase and Isocratic Elution

To test the effects of combining use of divalent ions and isocratic elution on the separation of full and empty capsid particles, samples of AAV8.1 material comprising full and empty capsid particles were run on CIMmultus® QA columns using one of three conditions: 1) NaCl, with gradient elution; 2) tetraethylammoniumchloride (TEAC) gradient and 2 mM MgCl₂, with gradient elution; and 3) tetraethylammoniumchloride (TEAC) and 2 mM MgCl₂, with isocratic elution.

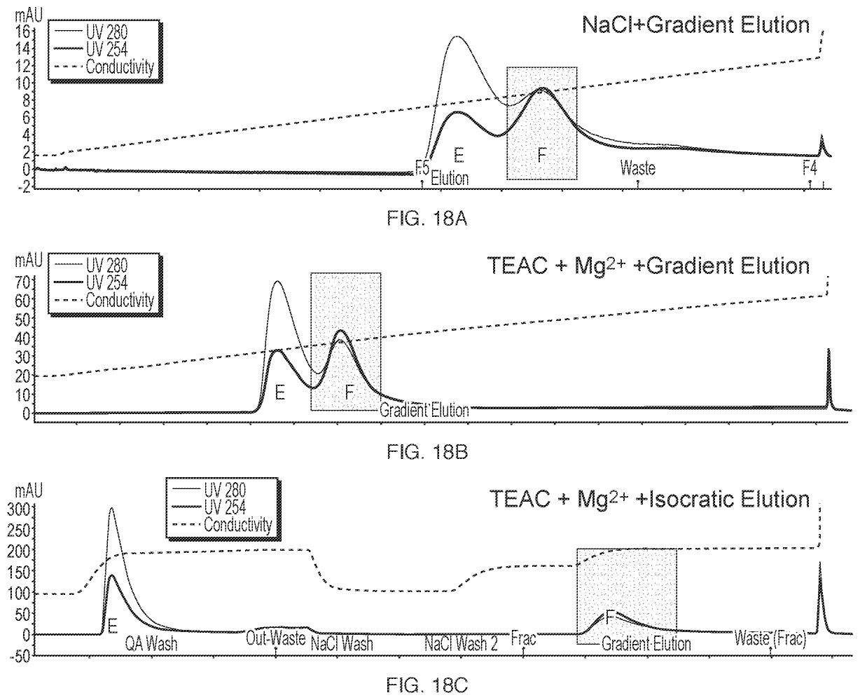

FIGS. 18A-18C depict representative chromatograms from these experiments. “E” denotes fractions corresponding to high relative amounts of empty capsids, and “F” denotes fractions corresponding to high relative amounts of full capsids.

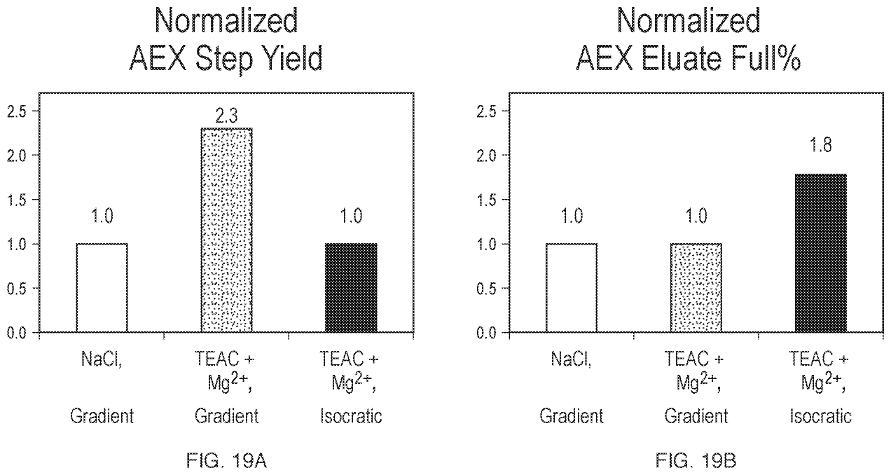

FIGS. 19A and 19B depict plots that show step yields (genomes of interest as measured by quantitative PCR) and percentages of full capsid particles (as measured by analytical ultracentrifugation) from these experiments, normalized to the levels for the NaCl gradient experiments.

As shown in FIG. 18A, using TEAC and MgCl₂ in a gradient elution method improved the step yield by 2.3-fold. However, as shown in FIG. 18B, using TEAC and MgCl₂ in an isocratic elution method improved the percentage of full capsid particles by 1.8-fold, while maintaining a step yield comparable to that of the step yield obtained with the NaCl gradient.

### Example 14: Use of Anionic Species in the Mobile Phase

To test the effects of using elution solutions comprising anionic species on the separation of full and empty capsid particles, samples of AAV8.1 material comprising full and empty capsid particles were run on CIMmultus® QA columns using one of several linear-gradient elution solutions.

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM NaCl, 2 mM MgCl₂, pH 9.0). The AAV particles were eluted from AEX media by a linear-gradient elution containing 20 mM BTP, 0.001% (w/v) Pluronic® F-68, 25-340 mM TEA-X at pH 9.0 over 90 column volumes (CVs), where X was one of the following counter-anion associated with tetraethylammonium (TEA): tetrafluoroborate (BF4), bromide (Br), and acetate (Ac). The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

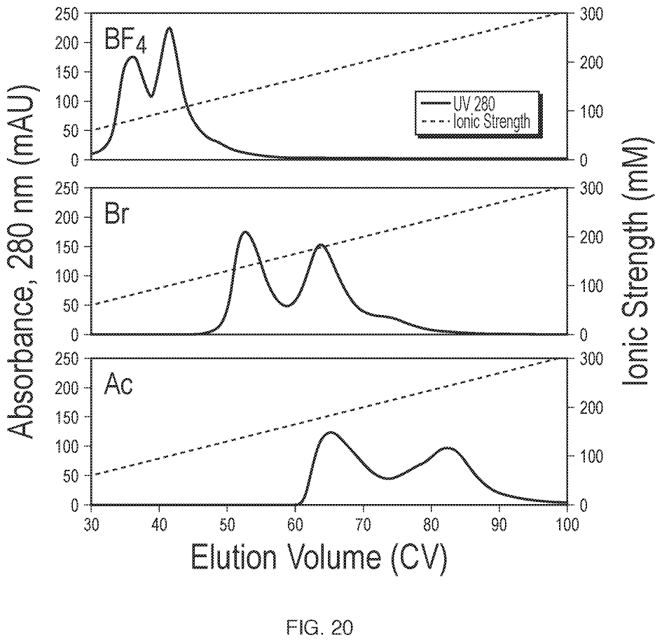

Representative results from these experiments are shown in FIG. 20. With all anionic species tested, two elution peaks were observed, corresponding to predominantly empty capsid particles (left-most peaks in each chromatogram) and predominantly full capsid particles (right-most peaks in each chromatogram). Compared to the elution peaks obtained when using TEA-BF₄ in the elution solution, the elution peaks obtained when using TEA-Br in the elution solution were more distinct, and the elution peaks obtained when using TEA-Ac in the elution solution were even more distinct.

Thus, this Example demonstrates that elution solutions comprising anionic species can be use in methods to separate empty capsid particles from full capsid particles.

### Example 15: Effect of MgCl₂ Concentration in a Gradient Elution Method

To test the effects of MgCl₂ concentration in the elution solution on the separation of full and empty capsid particles, samples of AAV8.1 material comprising full and empty capsid particles were run on CIMmultus® QA columns.

1.5 mL of affinity eluate was diluted 8-fold (to 15 mL) with 20 mM Bis-tris-propane (BTP) adjusted to pH 9.0 to reduce the load conductivity to approximately 4 mS/cm. Then 10 mL of the diluted affinity eluate was loaded on AEX media pre-equilibrated with pH-matched equilibration buffer (20 mM BTP, 25 mM NaCl, 2 mM MgCl₂, pH 9.0). The AAV particles were eluted from AEX media by a linear-gradient elution containing 20 mM BTP, 0.001% (w/v) Pluronic F-68@, 25-340 mM TEA-Ac at pH 9 over 90 column volumes (CVs) with or without MgCl₂, with the MgCl₂ concentration in the elution solution held constant at 0, 0.2 mM, 1 mM, or 5 mM. The column was then washed with 10 CVs of a high-salt strip solution (20 mM BTP, 2 M NaCl, 2 mM MgCl₂, pH 9.0), followed by 10 CVs of a sanitation solution (3 M NaCl, 1 M NaOH), and subsequently 10 CVs of a column-storage solution (50 mM Tris, 150 mM NaCl, 20% ethanol, pH 7.5).

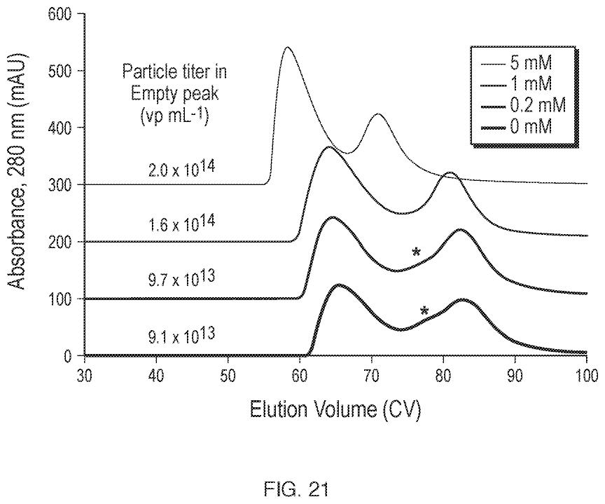

Representative resulting chromatograms are depicted in FIG. 21. At all concentrations tested, two elution peaks were observed, corresponding to predominantly empty capsid particles (left-most peaks in each chromatogram) and predominantly full capsid particles (right-most peaks in each chromatogram). However, at 0.2 mM and no MgCl₂, a shoulder peak was also detectable. This shoulder peak disappeared at the 1 mM and 5 mM MgCl₂ conditions tested. While the distances between the peaks decreased as the concentration of MgCl₂ was increased from 1 mM to 5 mM, the two peaks were still distinguishable at 5 mM MgCl₂.

### Example 16: Comparison of Isocratic Vs. Gradient Elution in Methods of Separating Empty and Full Capsid Particles

In this example, two methods were used to separate empty and full capsid particles: one using a gradient elution, and other using an isocratic elution. Both methods used at least one isocratic wash step with a solution comprising a quaternary ammonium salt.

Samples of AAV8.1 material comprising full and empty capsid particles were run on CIMmultus® QA columns using either Protocol 1 or Protocol 2 below:

### Protocol 1 (Gradient Elution):

Isocratic wash using a solution containing 20 mM BTP, 0.001% (w/v) Pluronic® F-68, and 206.5 mM TEA-Ac at pH 9 over 10 column-volumes
Linear gradient elution using a solution containing 20 mM BTP, 0.001% (w/v) Pluronic® F-68, 2 mM MgCl₂, and 25-182.5 mM NaCl at pH 9 over 30 column-volumes

### Protocol 2 (Isocratic Elution):

Isocratic wash using a solution containing 20 mM BTP, 0.001% (w/v) Pluronic® F-68, and 206.5 mM TEA-Ac at pH 9 over 10 column-volumes
Second isocratic wash using a solution containing 20 mM BTP, 0.001% (w/v) Pluronic® F-68, 2 mM MgCl₂, and 25 mM NaCl at pH 9 over 10 column-volumes
Isocratic elution using a solution containing 20 mM BTP, 0.001% (w/v) Pluronic® F-68, 2 mM MgCl₂, and 116.7 mM NaCl at pH 9 over 10 column-volumes

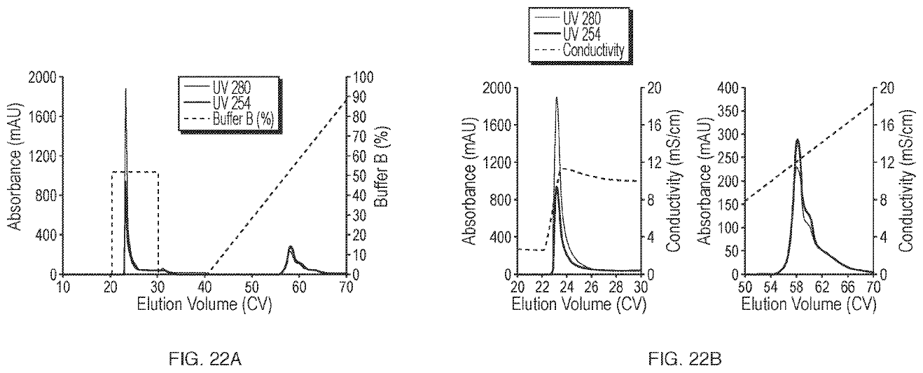

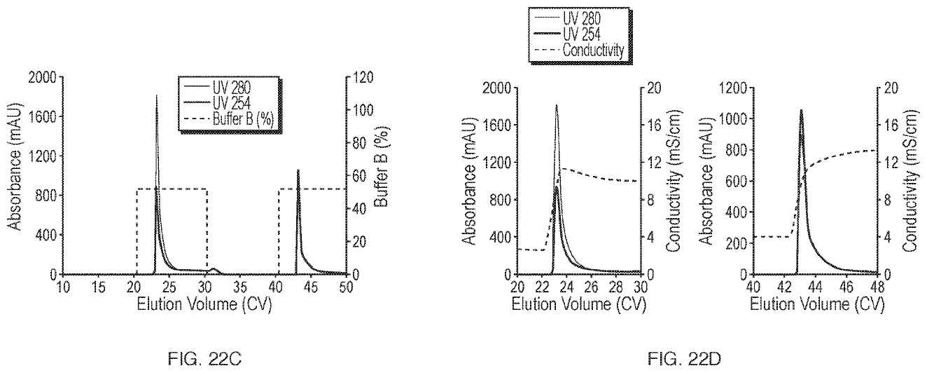

FIGS. 22A-22D depict representative results from these experiments. FIGS. 22A and 22C depict the chromatograms from the experiments using Protocol 1 (gradient elution) and Protocol 2 (isocratic elution), respectively. FIGS. 22B and 22D depict magnified images of the left peak (left panels in FIGS. 22B and 22D) and right peak (right panels in FIGS. 22B and 22D) from FIGS. 22A and 22C, respectively. The left-most peaks in FIGS. 22A and 22C correspond with predominantly empty capsid particles, and the right-most peaks in FIGS. 22A and 22C correspond with predominantly full capsid particles.

Both Protocols 1 and Protocols 2 resulted in clearly distinguishable peaks, allowing for separation of empty capsid particles from full capsid particles.

### Example 17: Trade-Off Between Percentage of Full Capsid Particles Eluted and Step Yield

The experiments described in this Example evaluate the trade-offs between the percentage of full capsid particles eluted and the step yield (genome recovery %) and compare the tradeoffs in methods that use an isocratic wash with a quaternary ammonium salt with methods that use NaCl in process buffers.

Samples of AAV8.1 material comprising full and empty capsid particles were run on CIMmultus® QA columns using either (1) NaCl process buffers or (2) isocratic washes using solutions comprising TEA-Ac. For both the NaCl process and isocratic TEA-Ac wash process, parallel sets of experiments were run using either isocratic or gradient elution with a solutions containing NaCl and MgCl₂. For the NaCl process experiments with an isocratic elution, both the isocratic and wash steps contained NaCl. For the NaCl process experiments with a gradient elution, the elution was operated in a gradient of NaCl, and no wash step was used.

Percentages of full capsid particles present in eluates were calculated using analytical ultra-centrifugation. Step yield, the amount of genome of interest in the pooled fraction relative to the amount in load material was determined by quantitative polymerase chain reaction (qPCR) methods.

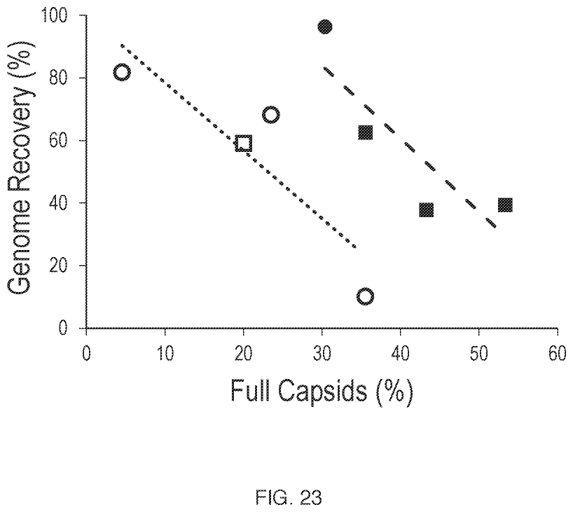

FIG. 23 depicts a plot of step yield (genome recovery %) (y-axis) against percentages of full capsid particles (x-axis) obtained from these experiments. The open markers denote data from anion exchange chromatography runs using NaCl process buffers, and closed markers denote data from anion exchange chromatography runs using an isocratic TEA-Ac wash. Circles denote data from runs that used isocratic elution, while squares denote data from runs that used gradient elution.

For both NaCl process runs and isocratic TEA-Ac wash process runs, a general trend appeared that indicated a trade-off between the percentage of full capsid particles recovered in eluates the step yield. However, both the step yield and the percentages of full capsid particles in eluates were better for the runs that used an isocratic TEA-Ac wash. (In FIG. 23, see the right-ward shift of the trend line for the solid circles and squares as compared to the trend line for the open circles and squares.)

Thus, the presently disclosed methods represent an improvement over previously disclosed methods of separating empty capsid particles from full capsid particles.

We claim:

1. A method of separating full capsid particles and empty capsid particles in a viral capsid preparation, the method comprising:
(a) applying the viral capsid preparation to an anion exchange medium;
(b) passing a first wash solution comprising a quaternary ammonium salt through the anion exchange medium to obtain a wash fraction comprising empty viral capsid particles;
(c) passing a second wash solution through the anion exchange medium to obtain a second wash fraction comprising the quaternary ammonium salt;
(d) passing an elution solution through the anion exchange medium to elute the full capsid particles; and
(e) collecting an elution fraction comprising full capsid particles;
thereby separating full capsid particles and empty capsid particles,
wherein (i) the concentration of the quaternary ammonium salt in the first wash solution remains constant throughout step (b) and/or (ii) the elution solution's salt composition remains constant throughout step (d).

2. The method of claim 1 comprising repeating step (b) one or more times.

3. The method of claim 1 or 2 comprising repeating step (c) one or more times.

4. The method of any one of claims 1-3 comprising repeating (d) one or more times.

5. An elution method comprising:
(a) contacting an anion-exchange medium with a viral capsid preparation comprising full capsid particles and empty capsid particles;
(b) applying to the anion-exchange medium a first wash solution comprising a quaternary ammonium salt to obtain a first wash fraction;
(c) applying to the anion-exchange medium a second wash solution, to obtain a second wash fraction; and
(d) applying to the anion-exchange medium an elution solution, and collecting an elution fraction,
wherein the concentration of the quaternary ammonium salt in the first wash solution remains constant throughout step (b) and/or (ii) the elution solution's salt composition remains constant throughout step (d).

6. The method of claim 5 comprising repeating step b) one or more times.

7. The method of claim 5 or 6 comprising repeating step c) one or more times.

8. The method of any one of claims 5-7 comprising repeating d) one or more times.

9. The method of any one of claims 1-8, wherein the second wash solution and the elution solution do not comprise a quaternary ammonium salt.

10. The method of claim 9, wherein, aside from the first wash solution, no other solution comprising the quaternary ammonium salt is passed through or applied to the anion exchange medium.

11. The method of claim 10, wherein, aside from the first wash solution, no other solution comprising any quaternary ammonium salt is passed through or applied to the anion exchange medium.

12. The method of any one of claims 1-11, wherein the first wash solution comprises a quaternary ammonium salt at a concentration of from about 90 mM to about 130 mM.

13. The method of claim 12, wherein the first wash solution comprises a concentration of about 110 mM quaternary ammonium salt.

14. The method of any one of claims 1-13, wherein the quaternary ammonium salt is a tetraalkylammonium chloride.

15. The method of claim 14, wherein the tetraalkylammonium chloride is selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof.

16. The method of claim 15, wherein the tetraalkylammonium chloride is TEAC.

17. The method of any one of claims 1-13, wherein the quaternary ammonium salt is a tetraalkylammonium acetate.

18. The method of claim 17, wherein the quaternary ammonium salt is selected from the group consisting of tetramethylammonium acetate, tetraethylammonium acetate (TEA-Ac), tetrapropylammonium acetate, tetrabutylammonium acetate, and any combination(s) thereof.

19. The method of claim 18, wherein the quaternary ammonium salt is TEA-Ac.

20. The method of any one of claims 1-13, wherein the quaternary ammonium salt is choline chloride.

21. The method of any one of claims 1-20, wherein any one or more of the first wash solution, the second wash solution, and/or the elution solution further comprise a divalent salt.

22. The method of claim 21, wherein the first wash solution further comprises a divalent salt.

23. The method of claim 22, wherein the divalent salt is MgCl₂.

24. The method of claim 23, wherein the first wash solution comprises MgCl₂ at a concentration from about 1 mM to about 10 mM.

25. The method of claim 24, wherein the first wash solution comprises MgCl₂ at a concentration of about 2 mM.

26. The method of claim 16, wherein the first wash solution comprises TEAC at a concentration from about 30 mM to about 200 mM.

27. The method of claim 26, wherein the first wash solution comprises TEAC at a concentration of about 110 mM.

28. The method of claim 27, wherein the first wash solution further comprises a divalent salt.

29. The method of claim 28, wherein the divalent salt is MgCl₂.

30. The method of claim 29, wherein the first wash solution comprises MgCl₂ at a concentration from about 1 mM to about 10 mM.

31. The method of claim 30, wherein the first wash solution comprises MgCl₂ at a concentration of about 2 mM.

32. The method of any one of claims 1-31, wherein the second wash solution and/or the elution solution comprises NaCl, Na₂SO₄, MgSO₄, or any combination thereof.

33. The method of claim 32, wherein the second wash solution comprises NaCl.

34. The method of claim 33, wherein the second wash solution comprises NaCl at a concentration from about 25 mM to about 375 mM.

35. The method of claim 34, wherein the second wash solution comprises NaCl at a concentration from about 70 mM to about 140 mM.

36. The method of any one of claims 1-35, wherein the elution solution comprises NaCl.

37. The method of claim 36, wherein the elution solution comprises NaCl at a concentration from about 25 mM to about 375 mM.

38. The method of claim 37, wherein the elution solution comprises NaCl at a concentration from about 90 mM to about 140 mM.

39. The method of any one of claims 1-35, wherein the elution solution comprises an anionic species.

40. The method of claim 39, wherein the anionic species is selected from the group consisting of tetrafluoroborate (BF₄), bromide (Br), and acetate (Ac).

41. The method of claim 39 or 40, wherein the anionic species is associated with tetraethylammonium (TEA).

42. The method of any one of claims 1-41, wherein the elution solution comprises MgCl₂.

43. The method of claim 42, wherein the elution solution comprises MgCl₂ at a concentration of at least 1 mM.

44. The method of claim 42, wherein the elution solution comprises MgCl₂ at a concentration of about 1 mM to about 5 mM.

45. The method of claim 42, wherein the elution solution comprises MgCl₂ at a concentration of about 1 mM, about 2 mM, about 3 mM, about 4 mM, or about 5 mM.

46. The method of any one of claims 1-45, wherein the first wash solution, the second wash solution, and/or the elution solution each comprise a buffer system at a pH of about 9.

47. The method of any one of claims 1-46, wherein the first wash solution elutes at least 60% of the empty capsid particles present in the viral capsid preparation.

48. The method of claim 47, wherein the first wash solution elutes at least 90% (e.g., from 90% to 100%, such as about 90%, 91%, 92%, 93%, 94%, 95%, 96%, 97%, 98%, 99%, or 100%) of the empty capsid particles present in the viral capsid preparation.

49. The method of any one of claims 1-48, wherein the first wash solution does not substantially elute the full capsid particles present in the viral capsid preparation.

50. The method of claim 49, wherein the first wash solution elutes 30% or less (e.g., from 0 to 30%, such as about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%) of the full capsid particles present in the viral capsid preparation.

51. The method of any one of claims 1-50, wherein the first wash solution elutes 1% or less of the full capsid particles present in the viral capsid preparation.

52. The method of any one of claims 1-51, wherein the second wash fraction comprises the quaternary ammonium salt.

53. The method of any one of claims 1-52, wherein the second wash solution elutes at least 50% of the quaternary ammonium salt present in the first wash solution.

54. The method of claim 53, wherein the second wash solution elutes at least 90% (e.g., from 90% to 100%, such as about 90%, 91%, 92%, 93%, 94%, 95%, 96%, 97%, 98%, 99%, or 100%) of the quaternary ammonium salt present in the first wash solution.

55. The method of claim 54, wherein the second wash solution elutes at least 99% of the quaternary ammonium salt present in the first wash solution.

56. The method of any one of claims 1-55, wherein the second wash solution does not substantially elute the full capsid particles present in the viral capsid preparation.

57. The method of claim 56, wherein the second wash solution elutes 20% or less (e.g., from 0 to 20%, such as about 0, 1%, 2%, 3%, 4%, 5%, 6%, 7%, 8%, 9%, 10%, 11%, 12%, 13%, 14%, 15%, 16%, 17%, 18%, 19%, or 20%) of the full capsid particles present in the viral capsid preparation.

58. The method of claim 57, wherein the second wash solution elutes 1% or less of the full capsid particles present in the viral capsid preparation.

59. The method of any one of claims 1-58, wherein the elution solution substantially elutes the full capsid particles present in the viral capsid preparation.

60. The method of claim 59, wherein the elution solution elutes at least 50% (e.g., from 50% to 100%, such as about 50%, 55%, 60%, 65%, 70%, 75%, 80%, 85%, 90%, 95%, or 100%) of the full capsid particles present in the viral capsid preparation.

61. The method of claim 60, wherein the elution solution elutes at least 90% (e.g., from 90% to 100%, such as about 90%, 91%, 92%, 93%, 94%, 95%, 96%, 97%, 98%, 99%, or 100%) of the full capsid particles present in the viral capsid preparation.

62. The method of claim 61, wherein the elution solution elutes at least 99% of the full capsid particles present in the viral capsid preparation.

63. The method of any one of claims 1-58, wherein the elution fraction comprises at least 50% of the full capsid particles present in the viral capsid preparation.

64. The method of claim 63, wherein the elution fraction comprises at least 90% (e.g., from 90% to 100%, such as about 90%, 91%, 92%, 93%, 94%, 95%, 96%, 97%, 98%, 99%, or 100%) of the full capsid particles present in the viral capsid preparation.

65. The method of claim 64, wherein the elution fraction comprises at least 99% of the full capsid particles present in the viral capsid preparation.

66. The method of any one of claims 1-65, wherein at least 35% (e.g., from 35% to 100%, such as about 35%, 40%, 45%, 50%, 55%, 60%, 65%, 70%, 75%, 80%, 85%, 90%, 95%, or 100%) of the capsid particles present in the elution fraction are full capsid particles.

67. The method of claim 66, wherein at least 40% (e.g., from 40% to 100%, such as about 40%, 45%, 50%, 55%, 60%, 65%, 70%, 75%, 80%, 85%, 90%, 95%, or 100%) of the capsid particles present in the elution fraction are full capsid particles.

68. The method of claim 67, wherein at least 45% (e.g., from 45% to 100%, such as about 40%, 45%, 50%, 55%, 60%, 65%, 70%, 75%, 80%, 85%, 90%, 95%, or 100%) of the capsid particles present in the elution fraction are full capsid particles.

69. The method of claim 68, wherein at least 50% (e.g., from 50% to 100%, such as about 50%, 55%, 60%, 65%, 70%, 75%, 80%, 85%, 90%, 95%, or 100%) of the capsid particles present in the elution fraction are full capsid particles.

70. The method of claim 69, wherein more than 50% (e.g., more than 50% and up to 100%, such as about 51%, 55%, 60%, 65%, 70%, 75%, 80%, 85%, 90%, 95%, or 100%) of the capsid particles present in the elution fraction are full capsid particles.

71. The method of any one of claims 1-70, wherein the elution fraction comprises no more than 50% of the empty capsid particles present in the viral capsid preparation, thereby substantially purifying the full capsid particles from the viral capsid preparation.

72. The method of claim 71, wherein the elution fraction comprises no more than 10% of the empty capsid particles present in the viral capsid preparation, thereby substantially purifying the full capsid particles from the viral capsid preparation.

73. The method of claim 72, wherein the elution fraction comprises no more than 1% of the empty capsid particles present in the viral capsid preparation, thereby substantially purifying the full capsid particles from the viral capsid preparation.

74. The method of any one of the previous claims, wherein the concentration of the quaternary ammonium salt in the first wash solution remains constant throughout step (b).

75. The method of claim 74, wherein the first wash solution's salt composition remains constant throughout step (b).

76. The method of any one of the previous claims, wherein the second wash solution's salt composition remains constant throughout step (c).

77. The method of any one of the previous claims, wherein the elution solution's salt composition remains constant throughout step (d).

78. The method of claim 77, wherein 1-20 total column volumes are eluted.

79. The method of claim 78, wherein 1-10 total column volumes are eluted.

80. The method of any one of the previous claims, wherein the first wash solution's salt composition, the second wash solution's salt composition, and the elution solution's salt composition are remaining constant throughout each individual wash or elution step.

81. The method of any one of claims 1-73, wherein the first wash solution's composition varies during step (b).

82. The method of any one of claims 1-73, wherein the second wash solution's composition varies during step (c).

83. The method of claim 82, wherein the concentration of a salt within the second wash increases continuously over time throughout step (c).

84. The method of claim 83, wherein the concentration of a salt within the second wash solution increases linearly over time throughout step (c).

85. The method of any one of claims 1-73, wherein the elution solution's composition varies during step (d).

86. The method of claim 85, wherein the concentration of a salt within the elution solution increases continuously over time throughout step (d).

87. The method of claim 86, wherein the concentration of a salt within the elution solution increases linearly over time throughout step (d).

88. The method of any one of claims 85-97, wherein 0-200 total column volumes are eluted.

89. The method of claim 88, wherein 50-150 total column volumes are eluted.

90. The method of claim 88, wherein about 90 total column volumes are eluted.

91. The method of any one of the previous claims, wherein the capsid is from AAV8 or a variant thereof.

92. The method of any one of the previous claims, wherein the anion exchange medium is a monolithic column.

93. The method of claim 92, wherein the monolithic column is a CIMmultus® QA column.

94. The method of any one of the preceding claims, wherein the method does not comprise using an isocratic elution gradient of MgCl₂.

95. The method of any one of the preceding claims, wherein, if the first wash solution further comprises MgCl₂ and the concentration of MgCl₂ is constant, the concentration of MgCl₂ is only constant throughout step (b).

96. A method of separating full capsid particles and empty capsid particles in a viral capsid preparation, the method comprising:
(a) applying the viral capsid preparation to an anion exchange medium;
(b) passing a first wash solution comprising a quaternary ammonium salt through the anion exchange medium to obtain a wash fraction comprising empty viral capsid particles;
(c) passing a second wash solution through the anion exchange medium to obtain a second wash fraction comprising the quaternary ammonium salt;
(d) passing an elution solution through the anion exchange medium to elute the full capsid particles; and
(e) collecting an elution fraction comprising full capsid particles;
thereby separating full capsid particles and empty capsid particles,
wherein the concentration of the quaternary ammonium salt in the first wash solution remains constant throughout step (b),
wherein the capsid is from AAV8 or a variant thereof, and
wherein at least 35% (e.g., from 35% to 100%, such as about 35%, 40%, 45%, 50%, 55%, 60%, 65%, 70%, 75%, 80%, 85%, 90%, 95%, or 100%) of the capsid particles present in the elution fraction are full capsid particles.

97. The method of claim 96, wherein the quaternary ammonium salt is a tetraalkylammonium chloride.

98. The method of claim 97, wherein the tetraalkylammonium chloride is selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof.

99. The method of claim 98, wherein the tetraalkylammonium chloride is TEAC.

100. The method of claim 96, wherein the quaternary ammonium salt is a tetraalkylammonium acetate.

101. The method of claim 100, wherein the quaternary ammonium salt is selected from the group consisting of tetramethylammonium acetate, tetraethylammonium acetate (TEA-Ac), tetrapropylammonium acetate, tetrabutylammonium acetate, and any combination(s) thereof.

102. The method of claim 101, wherein the quaternary ammonium salt is TEA-Ac.

103. The method of claim 96, wherein the quaternary ammonium salt is choline chloride.

104. A method for manufacturing full recombinant adeno-associated virus (rAAV) capsid particles, the method comprising the steps of:
(a) producing a viral capsid preparation comprising full rAAV capsid particles and empty rAAV capsid particles from cultured mammalian or insect cells;
(b) applying the viral capsid preparation to an anion exchange medium;
(c) passing a first wash solution comprising a quaternary ammonium salt through the anion exchange medium to obtain a wash fraction comprising empty rAAV viral capsid particles;
(d) passing a second wash solution through the anion exchange medium to obtain a second wash fraction comprising the quaternary ammonium salt;
(e) passing an elution solution through the anion exchange medium to elute the full rAAV capsid particles; and
(f) collecting an elution fraction comprising full rAAV capsid particles;
wherein (i) the concentration of the quaternary ammonium salt in the first wash solution remains constant throughout step (c) and/or (ii) the elution solution's salt composition remains constant throughout step (e).

105. A method for manufacturing full recombinant adeno-associated virus (rAAV) capsid particles, the method comprising the steps of:
(a) producing a viral capsid preparation comprising full rAAV capsid particles and empty rAAV capsid particles from cultured mammalian or insect cells;
(b) contacting an anion-exchange medium with the viral capsid preparation;
(c) applying to the anion-exchange medium a first wash solution comprising a quaternary ammonium salt to obtain a first wash fraction;
(d) applying to the anion-exchange medium a second wash solution, to obtain a second wash fraction; and
(e) applying to the anion-exchange medium an elution solution, and collecting an elution fraction,
wherein (i) the concentration of the quaternary ammonium salt in the first wash solution remains constant throughout step (c) and/or (ii) the elution solution's salt composition remains constant throughout step (e),
wherein the elution fraction comprises full rAAV capsid particles.

106. The method of claim 104 or 105, wherein the concentration of the quaternary ammonium salt in the first wash solution remains constant throughout step (c)

107. The method of claim 104, 105, or 106, wherein the rAAV capsid particles are rAAV8 capsid particles.

108. The method of any one of claims 104-107, wherein the quaternary ammonium salt is a tetraalkylammonium chloride.

109. The method of claim 108, wherein the tetraalkylammonium chloride is selected from the group consisting of tetramethylammonium chloride (TMAC), tetraethylammonium chloride (TEAC), tetrapropylammonium chloride (TPAC), tetrabutylammonium chloride (TBAC), benzyltributylammonium chloride (BTBAC), and any combination(s) thereof.

110. The method of claim 109, wherein the tetraalkylammonium chloride is TEAC.

111. The method of any one of claims 104-107, wherein the quaternary ammonium salt is a tetraalkylammonium acetate.

112. The method of claim 111, wherein the quaternary ammonium salt is selected from the group consisting of tetramethylammonium acetate, tetraethylammonium acetate (TEA-Ac), tetrapropylammonium acetate, tetrabutylammonium acetate, and any combination(s) thereof.

113. The method of claim 112, wherein the quaternary ammonium salt is TEA-Ac.

114. The method of any one of claims 104-107, wherein the quaternary ammonium salt is choline chloride.

115. The method of any one of claims 104-114, wherein at least 35% (e.g., 35% to 100%, such as about 35%, 40%, 45%, 50%, 55%, 60%, 65%, 70%, 75%, 80%, 85%, 90%, 95%, or 100%) of rAAV capsid particles in the elution fraction are full rAAV capsid particles.

116. A composition comprising full recombinant adeno-associated virus (rAAV) capsid particles, produced by a method of any one of claims 104-115.

117. The composition of claim 116, wherein at least 35% (e.g., 35% to 100%, such as about 35%, 40%, 45%, 50%, 55%, 60%, 65%, 70%, 75%, 80%, 85%, 90%, 95%, or 100%) of rAAV capsid particles in the composition are full rAAV capsid particles.
